(19) United States
(12) Patent Application Publication
(10) Pub. No.: US 2018/0133287 A1
(43) Pub. Date: May 17, 2018

(54) NUTRITIONAL COMPOSITIONS PROVIDING DIETARY MANAGEMENT OF COLIC

(71) Applicant: Mead Johnson Nutrition Company, Glenview, IL (US)

(72) Inventors: Lorenzo Morelli, Piacenza (IT); Valeria Sagheddu, Casella (IT); Peter van Dael, Newburgh, IN (US)

(21) Appl. No.: 15/350,538

(22) Filed: Nov. 14, 2016

(51) Int. Cl. A61K 38/17; A61K 35/745; A61K 36/889; A61K 36/48; A61K 35/20; A61K 31/202; A61K 31/685; A61K 31/688; A61K 31/683; A61K 31/7032; A61K 31/716; A61K 31/702; A61K 36/02; A61K 36/06; A61K 9/00; A23L 33/00

(52) U.S. Cl. CPC A61K 38/1709; A23V 2002/00; A61K 36/889; A61K 36/48; A61K 35/20; A61K 31/202; A61K 31/685; A61K 31/688; A61K 31/683; A61K 31/7032; A61K 31/716; A61K 31/702; A61K 36/02; A61K 36/06; A61K 9/0053; A23L 33/40; A61K 35/745

(57) ABSTRACT

A method for reducing the incidence of colic in a pediatric subject is presented, the method including administering to a subject a nutritional composition having about 1×10³ to about 1×10¹² cfu/100 kcal of LA metabolizing probiotic; up to about 7 g/100 kcal of a fat or lipid; up to about 5 g/100 kcal of a protein or protein equivalent source; about 0.06 g/100 kcal to about 1.5 g/100 kcal of enriched milk product; about 5 mg/100 kcal to about 90 mg/100 kcal of a source of long chain polyunsaturated fatty acids; and about 0.015 g/100 kcal to about 1.5 g/100 kcal of a prebiotic composition.

### TECHNICAL FIELD

The present disclosure relates to methods of managing colic in a pediatric subject through modifying the gut microbiome of the subject via administration of the nutritional composition disclosed herein. Some embodiments of the disclosure are directed to enhancing or promoting an increase in the concentration of beneficial bacteria in a pediatric subject, such as the Lactobacillus and Bifidobacterium species, while inhibiting the growth of bacterial species which can facilitate the onset of colic, especially the Blautia genus, including Ruminococcus gnavus.

Ruminococcus gnavus is an anaerobic Gram positive coccus, belonging to the Lachnospiraceae family (taxonomy of this species is under revision and it now believed that this species belong to the Blautia genus instead of Ruminococccus) that can be found in the gastrointestinal tracts of animals and humans; it is a non-butyrate producer, but it is a hydrogen producer and a mucin degrader. The increase of Blautia bacteria, especially Ruminococcus gnavus, in colicky infants is associated with a decrease of Bifidobacterium breve.

In some embodiments, the nutritional composition comprises a prebiotic blend which includes polydextrose and galacto-oligosaccharides. The disclosed nutritional compositions can also include an enriched milk product, such as an enriched whey protein concentrate (eWPC), and long chain polyunsaturated fatty acids, optionally also in combination with one or more of lactoferrin and short chain fatty acids.

### BACKGROUND ART

Infantile colic is painful to infants and can cause significant emotional distress and discomfort for parents, and as such is considered a major issue during infancy. Methods and compositions which can reduce or prevent infantile colic will provide relief for both infants and parents.

Infants with colic have been reported to have a different gut microbiota as compared to healthy infants, but no causal relationship has to date been established. However, it has now been suggested that the specificity of the modified gut microbiome of colicky infants, which is unable to metabolize dietary linoleic acid (LA) to conjugated linoleic acid (CLA), is associated with increased production of hydrogen and pro-inflammatory substances. Contrariwise, species of Bifidobacterium, such as Bifidobacterium breve, Bifidobacterium catenulatum, Bifidobacterium pseudocatenulatum, or the like, can metabolize LA to CLA and, thus, reduce hydrogen production. Member of the Blautia genus, such as Ruminococcus gnavus, cannot metabolize LA to CLA; as such, an abundance of the Bifidobacterium species, to the exclusion of the Blautia species, can reduce the production of hydrogen and other pro-inflammatory substances and reduce colic.

Recently, it has been found that increasing the ratio of bacteria capable of metabolizing the lipid fraction of a nutritional composition so as to convert LA into CLA and its derivatives (“LA metabolizing probiotic”) to the Blautia genus, including Ruminococcus gnavus, in the gut of a pediatric subject, especially an infant, will lead to improved metabolysis or conversion of LA to CLA and, thus, reduce the incidence of colic. The ratio of LA metabolizing probiotic to Blautia/Ruminococcus gnavus can be increased by increasing the presence of beneficial bacteria in the infant gut, such as by providing a nutritional composition which includes Bifidobacterium breve, preferably in combination with prebiotics such as polydextrose and galacto-oligosaccharides; and by decreasing the presence of Blautia bacteria in the gut, through the presence of competitive bacteria such as Bifidobacteria and/or Lactobacilli through competition for the same nutrients, by either supplementing with the CLA converting bacteria or by stimulating the growth of the CLA converting gut microbiota selectively with prebiotics. Accordingly, it would be beneficial to provide a nutritional composition for pediatric subjects, as well as pregnant or lactating women, that contains such a combination.

### BRIEF SUMMARY

Briefly, the present disclosure relates to methods of managing colic in pediatric subjects through modifying the gut microbiome of the subject via administration to the pediatric subject or a pregnant or lactating woman of a nutritional composition which can increase the ratio of LA metabolizing probiotic to the Blautia genus, including Ruminococcus gnavus, in the gut of the subject. In certain embodiments, the number of LA metabolizing probiotic, such as Bifidobacterium breve, is at least one log higher than the number of Blautia bacteria. In other embodiments, the ratio of LA metabolizing probiotic, such as Bifidobacterium breve, to Blautia bacteria is at least 8:1; in other embodiments, the ratio of LA metabolizing probiotic, such as Bifidobacterium breve, to Blautia bacteria is at least 10:1. In still other embodiments, the ratio of LA metabolizing probiotic, such as Bifidobacterium breve, to Blautia bacteria is at least 12:1.

In certain embodiments, the nutritional composition further comprises an enriched lipid fraction derived from milk, such as an enriched whey protein concentrate (eWPC). In some embodiments the nutritional composition may include an enriched lipid fraction derived from milk that includes milk fat globules. The addition of the milk fat globules provides an enriched fat and lipid source to the infant that may be more fully digested by a pediatric subject. The nutritional composition can also include long chain polyunsaturated fatty acids, optionally in combination with one or more of lactoferrin and short chain fatty acids in some embodiments.

In certain embodiments, the enriched lipid fraction and/or the milk fat globules may include saturated fatty acids, trans-fatty acids, monounsaturated fatty acids, polyunsaturated fatty acids, cholesterol, branched chain fatty acids “BCFAs”, conjugated linoleic acid “CLA”, phospholipids, or milk fat globule membrane protein, and mixtures thereof.

More particularly, in certain embodiments, the nutritional composition of the present disclosure comprises:
about 1×10³ to about 1×10¹² cfu/100 kcal of LA metabolizing probiotic;
up to about 7 g/100 kcal of a fat or lipid;
up to about 5 g/100 kcal of a protein or protein equivalent source;
about 0.06 g/100 kcal to about 1.5 g/100 kcal of enriched milk product;
about 5 mg/100 kcal to about 90 mg/100 kcal of long chain polyunsaturated fatty acids (“LCPUFAs”); and
about 0.015 g/100 kcal to about 1.5 g/100 kcal of a prebiotic.

In some embodiments, the LA metabolizing probiotic comprises Bifidobacterium breve. In other embodiments, the LA metabolizing probiotic comprises Bifidobacterium catenulatum or Bifidobacterium pseudocatenulatum. Moreover, the LA metabolizing probiotic can include combinations of Bifidobacterium breve, Bifidobacterium catenulatum, and Bifidobacterium pseudocatenulatum; combinations of Bifidobacterium breve and Bifidobacterium catenulatum; combinations of Bifidobacterium breve with Bifidobacterium pseudocatenulatum; or the combination of Bifidobacterium catenulatum with Bifidobacterium pseudocatenulatum.

Further, in some embodiments, the enriched milk product comprises an eWPC. Also, in some embodiments, the nutritional composition further comprises about 5 mg/100 kcal to about 300 mg/100 kcal of lactoferrin. The pediatric subject may be an infant, and the nutritional composition may be provided as an infant formula.

Enriched milk product means, in the context of the present disclosure, a milk product enriched with milk fat globule membrane (MFGM) components, such as MFGM proteins and lipids. The enriched milk product can be formed by, e.g., fractionation of non-human (e.g., bovine) milk. Enriched milk products have a total protein level which can range between 20% and 90%, more preferably between 68% and 80%, of which between 3% and 50% is MFGM proteins; in some embodiments, MFGM proteins make up from 7% to 13% of the enriched milk product protein content. Enriched milk products also comprise from 0.5% to 5% (and, at times, 1.2% to 2.8%) sialic acid, from 2% to 25% (and, in some embodiments, 4% to 10%) phospholipids, from 0.4% to 3% sphingomyelin, from 0.05% to 1.8%, and, in certain embodiments 0.10% to 0.3%, gangliosides and from 0.02% to about 1.2%, more preferably from 0.2% to 0.9%, cholesterol. Thus, enriched milk products include desirable components at levels higher than found in bovine and other non-human milks.

Milk, such as bovine milk, is a complex emulsion that contains several classes of components which fulfill nutritional requirements and/or deliver special health benefits to the consumer. The fat component of milk exists in the form of globules which range in size from 0.1 to 10 microns. The fat globules comprise about 98% triacylglycerols (TAGs, which are the major storage form of energy in animals) and are surrounded and stabilized by the milk fat globule membrane, which is derived from endoplasmic reticulum membrane and cell membrane.

The milk fat globule membrane is comprised of a trilayer lipid structure that includes a complex mixture of phospholipids, proteins, glycoproteins, triglycerides, polar lipids, cholesterol, enzymes and other components which are generally not abundant in conventional infant formulas and growing-up milks.

The polar lipids found in MFGM are composed of:
(i) Glycerophospholipids such as phosphatidylcholine (PC), phosphatidylethanolamine (PE), phosphatidylserine (PS), and phosphatidylinositol (PI), and their derivatives and
(ii) Sphingoids or sphingolipids such as sphingomyelin (SM) and glycosphingolipids comprising cerebrosides (neutral glycosphingolipids containing uncharged sugars) and the gangliosides (GG, acidic glycosphingolipids containing sialic acid) and their derivatives.

Phosphatidylethanolamine is a phospholipid found in biological membranes, particularly in nervous tissue such as the white matter of brain, nerves, neural tissue, and in spinal cord, where it makes up 45% of all phospholipids. Sphingomyelin is a type of sphingolipid found in animal cell membranes, especially in the membranous myelin sheath that surrounds some nerve cell axons. It usually consists of phosphocholine and ceramide, or a phosphoethanolamine head group; therefore, sphingomyelins can also be classified as sphingophospholipids. In humans, SM represents ˜85% of all sphingolipids, and typically makes up 10-20 mol % of plasma membrane lipids. Sphingomyelins are present in the plasma membranes of animal cells and are especially prominent in myelin, a membranous sheath that surrounds and insulates the axons of some neurons.

LCPUFAs such as docosahexaenoic acid (“DHA”) are omega-3 fatty acids that are a primary structural component of the human brain, cerebral cortex, skin, sperm, testicles and retina. DHA can be synthesized from alpha-linolenic acid or obtained directly from maternal milk or fish oil. DHA is the most abundant omega-3 fatty acid in the brain and retina. DHA comprises 40% of the polyunsaturated fatty acids (PUFAs) in the brain and 60% of the PUFAs in the retina. Fifty percent of the weight of a neuron's plasma membrane is composed of DHA. DHA is richly supplied during breastfeeding, and DHA levels can be high in human milk. DHA concentrations in human milk range from 0.07% to greater than 1.0% of total fatty acids, with a mean of about 0.32%. DHA levels in human milk are higher if a mother's diet is high in fish.

It is to be understood that both the foregoing general description and the following detailed description present embodiments of the disclosure and are intended to provide an overview or framework for understanding the nature and character of the disclosure as it is claimed. The description serves to explain the principles and operations of the claimed subject matter. Other and further features and advantages of the present disclosure will be readily apparent to those skilled in the art upon a reading of the following disclosure.

### BRIEF DESCRIPTION OF THE DRAWINGS

FIG. 1 illustrates the metabolomics profiles of fecal samples taken from a single subject during colic (upper double bars) and non-colic states (upper double boxes) in accordance with Example 1.

FIG. 2 illustrates the separation of the triplicates of feces of colicky babies (red) and controls (blue) analyzed by GC-MS using the PLSDA analysis (taking in consideration all compounds and all infants) in accordance with Example 2.

FIG. 3 reports the Denaturing Gradient Gel Electrophoresis (DGGE) output obtained when a specific bacterial population was analyzed; every band in the gel represents a different population and colicky infants harbor a more complex microbiota compared to control infants in accordance with Example 3.

FIG. 4 illustrates the presence of the Blautia genus, including Ruminococcus gnavus, (blue arrow) in 4 out of 7 and Ruminococcus gnavus (red arrow) in 6 out of 7 colicky infants and respectively in 1 out of 7 and in 3 out of 7 control infants in accordance with Example 3.

FIG. 5 illustrates the clustering analyses at the family level in accordance with Example 6.

FIG. 6 illustrates the clustering analyses at the species level supported a role for Ruminococcus gnavus in causing infantile colic (see violet bars for the presence of Ruminococcus gnavus in a number of samples, especially in samples 24, 25 and 32, which all are from colicky infants (Colicky samples are marked by a red circle: 10, 24, 25, 31, 32, 35, and 38) in accordance with Example 6.

FIG. 7 illustrates the differential abundances of species in the two groups; beside Ruminococcus gnavus also Haemophilus, Akkermansia and Bifidobacterium breve seem to differ deeply between the two groups in accordance with Example 6.

### DETAILED DESCRIPTION

Reference now will be made in detail to the embodiments of the present disclosure, one or more examples of which are set forth herein below. Each example is provided by way of explanation of the nutritional composition of the present disclosure and is not a limitation. In fact, it will be apparent to those skilled in the art that various modifications and variations can be made to the teachings of the present disclosure without departing from the scope of the disclosure. For instance, features illustrated or described as part of one embodiment, can be used with another embodiment to yield a still further embodiment.

Thus, it is intended that the present disclosure covers such modifications and variations as come within the scope of the appended claims and their equivalents. Other objects, features and aspects of the present disclosure are disclosed in or are obvious from the following detailed description. It is to be understood by one of ordinary skill in the art that the present discussion is a description of exemplary embodiments only and is not intended as limiting the broader aspects of the present disclosure.

The present disclosure relates generally to nutritional compositions that are suitable for administration to a pediatric subject, especially an infant. Alternatively, the disclosed nutritional compositions can be administered to a pregnant or lactating woman, so as to provide the described benefits to her infant. Additionally, the disclosure relates to methods of managing colic in pediatric subjects through modifying the gut microbiome of the subject via administration of the nutritional composition disclosed herein. Some embodiments of the disclosure are directed to enhancing or promoting an increase in the concentration of beneficial bacteria in a pediatric subject, such as the Lactobacillus and Bifidobacterium species, while inhibiting the growth of bacterial species which can facilitate or cause the development of colic, especially members of the Blautia genus, including Ruminococcus gnavus.

“Nutritional composition” means a substance or formulation that satisfies at least a portion of a subject's nutrient requirements. The terms “nutritional(s)”, “nutritional formula(s)”, “enteral nutritional(s)”, and “nutritional supplement(s)” are used as non-limiting examples of nutritional composition(s) throughout the present disclosure. Moreover, “nutritional composition(s)” may refer to liquids, powders, gels, pastes, solids, concentrates, suspensions, or ready-to-use forms of enteral formulas, oral formulas, formulas for infants, formulas for pediatric subjects, formulas for children, growing-up milks and/or formulas for adults.

“Infantile colic” or “colic” is defined as paroxysmal (sudden, brief and repetitive), excessive, inconsolable crying for more than three hours a day, at least three days a week, for one week or more in an otherwise healthy baby. It is most frequently observed in infants between two weeks and four months of age. It is recognized as a functional gastrointestinal disorder of infancy by the Rome III classification. Persistent infantile colic can contribute to parental fatigue and distress and may result in strained parental relationships, and poor parental engagement with their infant.

The term “enteral” means deliverable through or within the gastrointestinal, or digestive, tract. “Enteral administration” includes oral feeding, intragastric feeding, transpyloric administration, or any other administration into the digestive tract. “Administration” is broader than “enteral administration” and includes parenteral administration or any other route of administration by which a substance is taken into a subject's body.

“Pediatric subject” means a human no greater than 13 years of age. In some embodiments, a pediatric subject refers to a human subject that is between birth and 8 years old. In other embodiments, a pediatric subject refers to a human subject between 1 and 6 years of age. In still further embodiments, a pediatric subject refers to a human subject between 6 and 12 years of age. The term “pediatric subject” may refer to infants (preterm or full term) and/or children, as described below.

“Infant” means a human subject ranging in age from birth to not more than one year and includes infants from 0 to 12 months corrected age. The phrase “corrected age” means an infant's chronological age minus the amount of time that the infant was born premature. Therefore, the corrected age is the age of the infant if it had been carried to full term. The term infant includes low birth weight infants, very low birth weight infants, extremely low birth weight infants and preterm infants. “Preterm” means an infant born before the end of the 37th week of gestation. “Late preterm” means an infant form between the 34th week and the 36th week of gestation. “Full term” means an infant born after the end of the 37th week of gestation. “Low birth weight infant” means an infant born weighing less than 2500 grams (approximately 5 lbs, 8 ounces).

“Very low birth weight infant” means an infant born weighing less than 1500 grams (approximately 3 lbs, 4 ounces). “Extremely low birth weight infant” means an infant born weighing less than 1000 grams (approximately 2 lbs, 3 ounces).

“Child” means a subject ranging in age from 12 months to 13 years. In some embodiments, a child is a subject between the ages of 1 and 12 years old. In other embodiments, the terms “children” or “child” refer to subjects that are between one and about six years old, or between about seven and about 12 years old. In other embodiments, the terms “children” or “child” refer to any range of ages between 12 months and about 13 years.

The term “degree of hydrolysis” refers to the extent to which peptide bonds are broken by a hydrolysis method. The degree of protein hydrolysis for purposes of characterizing the hydrolyzed protein component of the nutritional composition is easily determined by one of ordinary skill in the formulation arts by quantifying the amino nitrogen to total nitrogen ratio (AN/TN) of the protein component of the selected formulation. The amino nitrogen component is quantified by USP titration methods for determining amino nitrogen content, while the total nitrogen component is determined by the Tecator Kjeldahl method, all of which are well known methods to one of ordinary skill in the analytical chemistry art.

The term “partially hydrolyzed” means having a degree of hydrolysis which is greater than 0% but less than about 50%.

The term “extensively hydrolyzed” means having a degree of hydrolysis which is greater than or equal to about 50%.

The term “protein-free” means containing no measurable amount of protein, as measured by standard protein detection methods such as sodium dodecyl (lauryl) sulfate-polyacrylamide gel electrophoresis (SDS-PAGE) or size exclusion chromatography. In some embodiments, the nutritional composition is substantially free of protein, wherein “substantially free” is defined herein below.

“Infant formula” means a composition that satisfies at least a portion of the nutrient requirements of an infant. In the United States, the content of an infant formula is dictated by the federal regulations set forth at 21 C.F.R. Sections 100, 106, and 107. These regulations define macronutrient, vitamin, mineral, and other ingredient levels in an effort to simulate the nutritional and other properties of human breast milk.

“Follow-up formula” means a composition that satisfies at least a portion of the nutrient requirements of infant from the 6th month onwards and for young children 1-3 years of age. The Codex Standard for Follow-Up Formula (CODEX STAN 156-1987) defines the compositional requirements of a follow-up formula in Section 3.

“Milk-based” means comprising at least one component that has been drawn or extracted from the mammary gland of a mammal. In some embodiments, a milk-based nutritional composition comprises components of milk that are derived from domesticated ungulates, ruminants or other mammals or any combination thereof.

Moreover, in some embodiments, milk-based means comprising bovine casein, whey, lactose, or any combination thereof. Further, “milk-based nutritional composition” may refer to any composition comprising any milk-derived or milk-based product known in the art.

“Milk” means a component that has been drawn or extracted from the mammary gland of a mammal. In some embodiments, the nutritional composition comprises components of milk that are derived from domesticated ungulates, ruminants or other mammals or any combination thereof.

“Fractionation procedure” includes any process in which a certain quantity of a mixture is divided up into a number of smaller quantities known as fractions. The fractions may be different in composition from both the mixture and other fractions. Examples of fractionation procedures include but are not limited to, melt fractionation, solvent fractionation, supercritical fluid fractionation and/or combinations thereof.

“Fat globule” refers to a small mass of fat surrounded by phospholipids and other membrane and/or serum proteins, where the fat itself can be a combination of any vegetable or animal fat.

“Polar lipids” are the main constituents of natural membranes, occurring in all living organisms. The polar lipids in milk (i.e., milk polar lipids) are mainly situated in the milk fat globule membrane. Polar lipids are also present in sources other than milk such as eggs, meat and plants.

Polar lipids are generally divided into phospholipids and sphingolipids (including gangliosides), which are amphiphilic molecules with a hydrophobic tail and a hydrophilic head group. The glycerophospholipids consist of a glycerol backbone on which two fatty acids are esterified on positions sn-1 and sn-2. These fatty acids are more unsaturated than the triglyceride fraction of milk. On the third hydroxyl, a phosphate residue with different organic groups (choline, serine, ethanolamine, etc.) may be linked. Generally, the fatty acid chain on the sn-1 position is more saturated compared with that at the sn-2 position. Lysophospholipids contain only one acyl group, predominantly situated at the sn-1 position. The head group remains similar. The characteristic structural unit of sphingolipids is the sphingoid base, a long-chain (12-22 carbon atoms) aliphatic amine containing two or three hydroxyl groups. Sphingosine (d18:1), is the most prevalent sphingoid base in mammalian sphingolipids, containing 18 carbon atoms, two hydroxyl groups and one double bond. A ceramide is formed when the amino group of this sphingoid base is linked with, usually, a saturated fatty acid. On this ceramide unit, an organophosphate group can be bound to form a sphingophospholipid (e.g., phosphocholine in the case of sphingomyelin) or a saccharide to form the sphingoglycolipids (glycosylceramides). Monoglycosylceramides, like glucosylceramide or galactosylceramide are often denoted as cerebrosides while tri- and tetraglycosylceramides with a terminal galactosamine residue are denoted as globosides. Finally, gangliosides are highly complex oligoglycosylceramides, containing one or more sialic acid groups in addition to glucose, galactose and galactosamine.

“Nutritionally complete” means a composition that may be used as the sole source of nutrition, which would supply essentially all of the required daily amounts of vitamins, minerals, and/or trace elements in combination with proteins, carbohydrates, and lipids. Indeed, “nutritionally complete” describes a nutritional composition that provides adequate amounts of carbohydrates, lipids, essential fatty acids, proteins, essential amino acids, conditionally essential amino acids, vitamins, minerals and energy required to support normal growth and development of a subject.

Therefore, a nutritional composition that is “nutritionally complete” for a preterm infant will, by definition, provide qualitatively and quantitatively adequate amounts of carbohydrates, lipids, essential fatty acids, proteins, essential amino acids, conditionally essential amino acids, vitamins, minerals, and energy required for growth of the preterm infant.

A nutritional composition that is “nutritionally complete” for a full term infant will, by definition, provide qualitatively and quantitatively adequate amounts of all carbohydrates, lipids, essential fatty acids, proteins, essential amino acids, conditionally essential amino acids, vitamins, minerals, and energy required for growth of the full term infant.

A nutritional composition that is “nutritionally complete” for a child will, by definition, provide qualitatively and quantitatively adequate amounts of all carbohydrates, lipids, essential fatty acids, proteins, essential amino acids, conditionally essential amino acids, vitamins, minerals, and energy required for growth of a child.

As applied to nutrients, the term “essential” refers to any nutrient that cannot be synthesized by the body in amounts sufficient for normal growth and to maintain health and that, therefore, must be supplied by the diet. The term “conditionally essential” as applied to nutrients means that the nutrient must be supplied by the diet under conditions when adequate amounts of the precursor compound is unavailable to the body for endogenous synthesis to occur.

“Probiotic” means a live microorganism which when consumed in adequate amounts as part of food confer a health benefit on the host. A probiotic should also be scientifically substantiated as being safe for use by humans.

The term “inactivated probiotic” means a probiotic wherein the metabolic activity or reproductive ability of the referenced probiotic has been reduced or destroyed. The “inactivated probiotic” does, however, still retain, at the cellular level, its cell structure or other structure associated with the cell, for example exopolysaccharide and at least a portion its biological glycol-protein and DNA/RNA structure. As used herein, the term “inactivated” is synonymous with “non-viable”.

“Prebiotic” means a non-digestible food ingredient that beneficially affects the host by selectively stimulating the growth and/or activity of one or a limited number of bacteria in the digestive tract that can improve the health of the host.

“Branched Chain Fatty Acid” (“BCFA”) means a fatty acid containing a carbon constituent branched off the carbon chain. Typically the branch is an alkyl branch, especially a methyl group, but ethyl and propyl branches are also known. The addition of the methyl branch lowers the melting point compared with the equivalent straight chain fatty acid. This includes branched chain fatty acids with an even number of carbon atoms in the carbon chain. Examples of these can be isomers of tetradecanoic acid, hexadecanoic acid.

“Trans-fatty acid” means an unsaturated fat with a trans-isomer. Trans-fats may be monounsaturated or polyunsaturated. Trans refers to the arrangement of the two hydrogen atoms bonded to the carbon atoms involved in a double bond. In the trans arrangement, the hydrogens are on opposite sides of the bond. Thus a trans-fatty acid is a lipid molecule that contains one or more double bonds in trans geometric configuration.

“Phospholipids” means an organic molecule that contains a diglyceride, a phosphate group and a simple organic molecule. Examples of phospholipids include but are not limited to, phosphatidic acid, phosphatidylethanolamine, phosphatidylcholine, phosphatidylserine, phosphatidylinositol, phosphatidylinositol phosphate, phosphatidylinositol biphosphate and phosphatidylinositol triphosphate, ceramide phosphorylcholine, ceramide phosphorylethanolamine and ceramide phosphorylglycerol. This definition further includes sphingolipids, glycolipids, and gangliosides.

“Phytonutrient” means a chemical compound that occurs naturally in plants. Phytonutrients may be included in any plant-derived substance or extract. The term “phytonutrient(s)” encompasses several broad categories of compounds produced by plants, such as, for example, polyphenolic compounds, anthocyanins, proanthocyanidins, and flavan-3-ols (i.e. catechins, epicatechins), and may be derived from, for example, fruit, seed or tea extracts. Further, the term phytonutrient includes all carotenoids, phytosterols, thiols, and other plant-derived compounds. Moreover, as a skilled artisan will understand, plant extracts may include phytonutrients, such as polyphenols, in addition to protein, fiber or other plant-derived components. Thus, for example, apple or grape seed extract(s) may include beneficial phytonutrient components, such as polyphenols, in addition to other plant-derived substances.

“β-glucan” means all β-glucan, including specific types of β-glucan, such as (3-1,3-glucan or β-1,3;1,6-glucan. Moreover, β-1,3;1,6-glucan is a type of β-1,3-glucan. Therefore, the term “β-1,3-glucan” includes β-1,3;1,6-glucan.

“Pectin” means any naturally-occurring oligosaccharide or polysaccharide that comprises galacturonic acid that may be found in the cell wall of a plant. Different varieties and grades of pectin having varied physical and chemical properties are known in the art. Indeed, the structure of pectin can vary significantly between plants, between tissues, and even within a single cell wall. Generally, pectin is made up of negatively charged acidic sugars (galacturonic acid), and some of the acidic groups are in the form of a methyl ester group. The degree of esterification of pectin is a measure of the percentage of the carboxyl groups attached to the galactopyranosyluronic acid units that are esterified with methanol.

Pectin having a degree of esterification of less than 50% (i.e., less than 50% of the carboxyl groups are methylated to form methyl ester groups) are classified as low-ester, low methoxyl, or low methylated (“LM”) pectins, while those having a degree of esterification of 50% or greater (i.e., more than 50% of the carboxyl groups are methylated) are classified as high-ester, high methoxyl or high methylated (“HM”) pectins. Very low (“VL”) pectins, a subset of low methylated pectins, have a degree of esterification that is less than approximately 15%.

As used herein, “lactoferrin from a non-human source” means lactoferrin which is produced by or obtained from a source other than human breast milk. For example, lactoferrin for use in the present disclosure includes human lactoferrin produced by a genetically modified organism as well as non-human lactoferrin. The term “organism”, as used herein, refers to any contiguous living system, such as animal, plant, fungus or micro-organism.

As used herein, “non-human lactoferrin” means lactoferrin that has an amino acid sequence that is different than the amino acid sequence of human lactoferrin.

“Pathogen” means an organism that causes a disease state or pathological syndrome. Examples of pathogens may include bacteria, viruses, parasites, fungi, microbes or combination(s) thereof.

“Modulate” or “modulating” means exerting a modifying, controlling and/or regulating influence. In some embodiments, the term “modulating” means exhibiting an increasing or stimulatory effect on the level/amount of a particular component. In other embodiments, “modulating” means exhibiting a decreasing or inhibitory effect on the level/amount of a particular component.

All percentages, parts and ratios as used herein are by weight of the total formulation, unless otherwise specified.

All amounts specified as administered “per day” may be delivered in one unit dose, in a single serving or in two or more doses or servings administered over the course of a 24 hour period.

The nutritional composition of the present disclosure may be substantially free of any optional or selected ingredients described herein, provided that the remaining nutritional composition still contains all of the required ingredients or features described herein. In this context, and unless otherwise specified, the term “substantially free” means that the selected composition may contain less than a functional amount of the optional ingredient, typically less than 0.1% by weight, and also, including zero percent by weight of such optional or selected ingredient.

All references to singular characteristics or limitations of the present disclosure shall include the corresponding plural characteristic or limitation, and vice versa, unless otherwise specified or clearly implied to the contrary by the context in which the reference is made.

All combinations of method or process steps as used herein can be performed in any order, unless otherwise specified or clearly implied to the contrary by the context in which the referenced combination is made.

The methods and compositions of the present disclosure, including components thereof, can comprise, consist of, or consist essentially of the essential elements and limitations of the embodiments described herein, as well as any additional or optional ingredients, components or limitations described herein or otherwise useful in nutritional compositions.

As used herein, the term “about” should be construed to refer to both of the numbers specified as the endpoint(s) of any range. Any reference to a range should be considered as providing support for any subset within that range.

The present disclosure is directed to nutritional compositions which can increase the ratio of LA metabolizing probiotic to Ruminococcus gnavus in the gut of the subject. In an embodiment, the nutritional compositions are infant formulas which include a LA metabolizing probiotic and a prebiotic composition including galacto-oligosaccharides (GOS) and polydextrose (PDX). The nutritional compositions of the present disclosure support overall health and development in a pediatric human subject, such as an infant (preterm and/or term), and can prevent or reduce colic.

The unique combination of nutrients in the disclosed nutritional composition is believed to be capable of providing novel and unexpected benefits for infants in reduction of colic, as well as providing relief for parents of colicky infants.

The combination of nutrients in the nutritional composition combine in synergistic ways to provide the foregoing benefits. For instance, providing a LA metabolizing probiotic with a prebiotic comprising polydextrose and galacto-oligosaccharides can synergistically increase specific beneficial species of bacteria in the gastrointestinal tract, including Bifidobacterium species such as Bifidobacterium breve while also competitively reducing the presence of non-LA metabolizing bacteria such as Ruminococcus gnavus. This increase in the ratio of B. breve or other LA metabolizing probiotic to Ruminococcus gnavus results in a notable increase in the production of CLA from LA in the infant gut, leading to a marked decrease in colic. The presence of Bifidobacterium breve or other LA metabolizing probiotic(s) and the Blautia genus, including Ruminococcus gnavus, in the gut of an infant (and, thus, the way to measure the ratio of one to the other) can be determined using fecal samples from the infant, by analysis of fecal microbiota. Protocols used to extract DNA from fecal samples of infants, DGGE analysis and Quantitative PCR are those described by Sagheddu et al, 2016.DNA extraction for sequencing and sequence analysis have been performed according to Payne A N, Chassard C, Banz Y, Lacroix C. The composition and metabolic activity of child gut microbiota demonstrate differential adaptation to varied nutrient loads in an in vitro model of colonic fermentation. FEMS Microbiol Ecol (2012) 80:608-23. doi:10.1111/j.1574-6941.2012.01330.x

Other probiotics can also be included in the nutritional composition of the present disclosure, in order to compete with the Blautia species for nutrients, and thus reduce the presence of the Blautia bacteria in the gut; these may be selected from Bifidobacterium species or Lactobacillus species, and can include Lactobacillus rhamnosus GG (LGG) (ATCC number 53103), Bifidobacterium species other than Bifidobacterium breve, such as Bifidobacterium longum BB536 (BL999, ATCC: BAA-999), Bifidobacterium longum AH1206 (NCIMB: 41382), Bifidobacterium infantis 35624 (NCIMB: 41003), and Bifidobacterium animalis subsp. lactis BB-12 (DSM No. 10140), or any combination thereof.

The daily amount of LA metabolizing probiotic to be administered to a pediatric subject, or to a pregnant or lactating woman, may in some embodiments vary from about 1×10⁴ to about 1×10¹¹ cfu. In certain embodiments, the nutritional composition of the present disclosure may include LA metabolizing probiotic at a level of about 1×10⁴ to about 1×10¹¹ cfu per 100 g of powder (when the nutritional composition is provided in powder form for later reconstitution). Or, in embodiments, the nutritional composition of the present disclosure may include LA metabolizing probiotic at a level of about 1×10³ to about 1×10¹² cfu of probiotic(s) per 100 kcal. In other embodiments, the nutritional composition of the present disclosure may include LA metabolizing probiotic at a level of about 1×10⁴ to about 1×10¹⁰ cfu of probiotic(s) per 100 kcal; in yet other embodiments, LA metabolizing probiotic is present at a level of about 1×10⁶ to about 1×10⁹ cfu per 100 kcal.

Moreover, since the probiotic comprises viable cells, it may be desirable to provide a protective matrix for the probiotic to ensure continued viability during processing, storage and transport, by blending together at least one phospholipid and at least one glyceride; combining the probiotic, the protective matrix and water to produce a mixture; and drying the mixture of step to a final moisture content of about 4% or less. This method may comprise the additional step of adding the dried mixture to a powdered nutritional product or enclosing the dried mixture in a capsule.

As noted, the nutritional composition also contains one or more prebiotics (also referred to as a prebiotic component) in certain embodiments. Prebiotics exert health benefits, which may include, but are not limited to, selective stimulation of the growth and/or activity of one or a limited number of beneficial gut bacteria such as the LA metabolizing probiotic, stimulation of the growth and/or activity of ingested probiotic microorganisms, selective reduction in gut pathogens, and favorable influence on gut short chain fatty acid profile. Such prebiotics may be naturally-occurring, synthetic, or developed through the genetic manipulation of organisms and/or plants, whether such new source is now known or developed later. Prebiotics useful in the present disclosure may include oligosaccharides, polysaccharides, and other prebiotics that contain fructose, xylose, soya, galactose, glucose and mannose.

More specifically, prebiotics useful in the present disclosure may include polydextrose, polydextrose powder, lactulose, lactosucrose, raffinose, gluco-oligosaccharide, inulin, fructo-oligosaccharide, isomalto-oligosaccharide, soybean oligosaccharides, lactosucrose, xylo-oligosaccharide, chito-oligosaccharide, manno-oligosaccaride, aribino-oligosaccharide, siallyl-oligosaccharide, fuco-oligosaccharide, galacto-oligosaccharides and gentio-oligosaccharides.

In an embodiment, the total amount of prebiotics present in the nutritional composition may be from about 1.0 g/L to about 10.0 g/L of the composition. More preferably, the total amount of prebiotics present in the nutritional composition may be from about 2.0 g/L and about 8.0 g/L of the composition. In some embodiments, the total amount of prebiotics present in the nutritional composition may be from about 0.01 g/100 kcal to about 1.5 g/100 kcal. In certain embodiments, the total amount of prebiotics present in the nutritional composition may be from about 0.15 g/100 kcal to about 1.5 g/100 kcal. Moreover, the nutritional composition may comprise a prebiotic component comprising polydextrose. In some embodiments, the prebiotic component comprises at least 20% w/w polydextrose or a mixture thereof.

The amount of polydextrose in the nutritional composition may, in an embodiment, be within the range of from about 0.015 g/100 kcal to about 1.5 g/100 kcal. In another embodiment, the amount of polydextrose is within the range of from about 0.2 g/100 kcal to about 0.6 g/100 kcal. In some embodiments, polydextrose may be included in the nutritional composition in an amount sufficient to provide between about 1.0 g/L and 10.0 g/L. In another embodiment, the nutritional composition contains an amount of polydextrose that is between about 2.0 g/L and 8.0 g/L. And in still other embodiments, the amount of polydextrose in the nutritional composition may be from about 0.05 g/100 kcal to about 1.5 g/100 kcal.

The prebiotic component also comprises galacto-oligosaccharides. The amount of galacto-oligosaccharides in the nutritional composition may, in an embodiment, be from about 0.015 g/100 kcal to about 1.0 g/100 kcal. In another embodiment, the amount of galacto-oligosaccharides in the nutritional composition may be from about 0.2 g/100 kcal to about 0.5 g/100 kcal.

In a particular embodiment of the present invention, polydextrose is administered in combination with galacto-oligosaccharides.

In a particular embodiment, galacto-oligosaccharides and polydextrose are supplemented into the nutritional composition in a total amount of at least about 0.015 g/100 kcal or about 0.015 g/100 kcal to about 1.5 g/100 kcal. In some embodiments, the nutritional composition may comprise galacto-oligosaccharides and polydextrose in a total amount of from about 0.1 to about 1.0 g/100 kcal.

In certain embodiments, lactoferrin from a non-human source is also included in the nutritional composition of the present disclosure. Lactoferrins are single chain polypeptides of about 80 kD containing 1-4 glycans, depending on the species. The 3-D structures of lactoferrin of different species are very similar, but not identical. Each lactoferrin comprises two homologous lobes, called the N- and C-lobes, referring to the N-terminal and C-terminal part of the molecule, respectively. Each lobe further consists of two sub-lobes or domains, which form a cleft where the ferric ion (Fe³+) is tightly bound in synergistic cooperation with a (bi)carbonate anion. These domains are called N1, N2, C1 and C2, respectively. The N-terminus of lactoferrin has strong cationic peptide regions that are responsible for a number of important binding characteristics. Lactoferrin has a very high isoelectric point (˜pl 9) and its cationic nature plays a major role in its ability to defend against bacterial, viral, and fungal pathogens. There are several clusters of cationic amino acids residues within the N-terminal region of lactoferrin mediating the biological activities of lactoferrin against a wide range of microorganisms. For instance, the N-terminal residues 1-47 of human lactoferrin (1-48 of bovine lactoferrin) are critical to the iron-independent biological activities of lactoferrin. In human lactoferrin, residues 2 to 5 (RRRR) and 28 to 31 (RKVR) are arginine-rich cationic domains in the N-terminus especially critical to the antimicrobial activities of lactoferrin. A similar region in the N-terminus is found in bovine lactoferrin (residues 17 to 42; FKCRRWQWRMKKLGAPSITCVRRAFA).

Lactoferrins from different host species may vary in their amino acid sequences though commonly possess a relatively high isoelectric point with positively charged amino acids at the end terminal region of the internal lobe. Suitable non-human lactoferrins for use in the present disclosure include, but are not limited to, those having at least 48% homology with the amino acid sequence of human lactoferrin. For instance, bovine lactoferrin (“bLF”) has an amino acid composition which has about 70% sequence homology to that of human lactoferrin. In some embodiments, the non-human lactoferrin has at least 55% homology with human lactoferrin and in some embodiments, at least 65% homology. Non-human lactoferrins acceptable for use in the present disclosure include, without limitation, bLF, porcine lactoferrin, equine lactoferrin, buffalo lactoferrin, goat lactoferrin, murine lactoferrin and camel lactoferrin.

In one embodiment, lactoferrin from a non-human source is present in the nutritional composition in an amount of at least about 15 mg/100 kcal. In certain embodiments, the nutritional composition may include between about 15 and about 300 mg lactoferrin per 100 kcal. In another embodiment, where the nutritional composition is an infant formula, the nutritional composition may comprise lactoferrin in an amount of from about 60 mg to about 150 mg lactoferrin per 100 kcal; in yet another embodiment, the nutritional composition may comprise about 60 mg to about 100 mg lactoferrin per 100 kcal.

In some embodiments, the nutritional composition can include lactoferrin in the quantities of from about 0.5 mg to about 1.5 mg per milliliter of formula. In nutritional compositions replacing human milk, lactoferrin may be present in quantities of from about 0.6 mg to about 1.3 mg per milliliter of formula. In certain embodiments, the nutritional composition may comprise between about 0.1 and about 2 grams lactoferrin per liter. In some embodiments, the nutritional composition includes between about 0.6 and about 1.5 grams lactoferrin per liter of formula.

The bLF that is used in certain embodiments may be any bLF isolated from whole milk and/or having a low somatic cell count, wherein “low somatic cell count” refers to a somatic cell count less than 200,000 cells/mL. By way of example, suitable bLF is available from Tatua Co-operative Dairy Co. Ltd., in Morrinsville, New Zealand, from FrieslandCampina Domo in Amersfoort, Netherlands or from Fonterra Co-Operative Group Limited in Auckland, New Zealand.

Lactoferrin from a non-human source for use in the present disclosure may be, for example, isolated from the milk of a non-human animal or produced by a genetically modified organism. For example, in U.S. Pat. No. 4,791,193, incorporated by reference herein in its entirety, Okonogi et al. discloses a process for producing bovine lactoferrin in high purity. Generally, the process as disclosed includes three steps. Raw milk material is first contacted with a weakly acidic cationic exchanger to absorb lactoferrin followed by the second step where washing takes place to remove nonabsorbed substances. A desorbing step follows where lactoferrin is removed to produce purified bovine lactoferrin. Other methods may include steps as described in U.S. Pat. Nos. 7,368,141, 5,849,885, 5,919,913 and 5,861,491, the disclosures of which are all incorporated by reference in their entirety.

In certain embodiments, lactoferrin utilized in the present disclosure may be provided by an expanded bed absorption (“EBA”) process for isolating proteins from milk sources. EBA, also sometimes called stabilized fluid bed adsorption, is a process for isolating a milk protein, such as lactoferrin, from a milk source comprises establishing an expanded bed adsorption column comprising a particulate matrix, applying a milk source to the matrix, and eluting the lactoferrin from the matrix with an elution buffer comprising about 0.3 to about 2.0 M sodium chloride. Any mammalian milk source may be used in the present processes, although in particular embodiments, the milk source is a bovine milk source. The milk source comprises, in some embodiments, whole milk, reduced fat milk, skim milk, whey, casein, or mixtures thereof.

In particular embodiments, the target protein is lactoferrin, though other milk proteins, such as lactoperoxidases or lactalbumins, also may be isolated. In some embodiments, the process comprises the steps of establishing an expanded bed adsorption column comprising a particulate matrix, applying a milk source to the matrix, and eluting the lactoferrin from the matrix with about 0.3 to about 2.0M sodium chloride. In other embodiments, the lactoferrin is eluted with about 0.5 to about 1.0 M sodium chloride, while in further embodiments, the lactoferrin is eluted with about 0.7 to about 0.9 M sodium chloride.

The expanded bed adsorption column can be any known in the art, such as those described in U.S. Pat. Nos. 7,812,138, 6,620,326, and 6,977,046, the disclosures of which are hereby incorporated by reference herein. In some embodiments, a milk source is applied to the column in an expanded mode, and the elution is performed in either expanded or packed mode. In particular embodiments, the elution is performed in an expanded mode. For example, the expansion ratio in the expanded mode may be about 1 to about 3, or about 1.3 to about 1.7. EBA technology is further described in international published application nos. WO 92/00799, WO 02/18237, WO 97/17132, which are hereby incorporated by reference in their entireties.

The isoelectric point of lactoferrin is approximately 8.9. Prior EBA methods of isolating lactoferrin use 200 mM sodium hydroxide as an elution buffer. Thus, the pH of the system rises to over 12, and the structure and bioactivity of lactoferrin may be comprised, by irreversible structural changes. It has now been discovered that a sodium chloride solution can be used as an elution buffer in the isolation of lactoferrin from the EBA matrix. In certain embodiments, the sodium chloride has a concentration of about 0.3 M to about 2.0 M. In other embodiments, the lactoferrin elution buffer has a sodium chloride concentration of about 0.3 M to about 1.5 M, or about 0.5 m to about 1.0 M.

The nutritional composition of the disclosure can, in some embodiments, also contain a source of LCPUFAs; especially a source of LCPUFAs that comprises docosahexaenoic acid. Other suitable LCPUFAs include, but are not limited to, α-linoleic acid, γ-linoleic acid, linoleic acid, linolenic acid, eicosapentaenoic acid (EPA) and arachidonic acid (ARA).

In an embodiment, especially if the nutritional composition is an infant formula, the nutritional composition is supplemented with both DHA and ARA. In this embodiment, the weight ratio of ARA:DHA may be between about 1:3 and about 9:1. In a particular embodiment, the ratio of ARA:DHA is from about 1:2 to about 4:1.

The amount of long chain polyunsaturated fatty acid in the nutritional composition is advantageously at least about 5 mg/100 kcal, and may vary from about 5 mg/100 kcal to about 100 mg/100 kcal, more preferably from about 10 mg/100 kcal to about 50 mg/100 kcal.

The nutritional composition may be supplemented with oils containing DHA and/or ARA using standard techniques known in the art. For example, DHA and ARA may be added to the composition by replacing an equivalent amount of an oil, such as high oleic sunflower oil, normally present in the composition. As another example, the oils containing DHA and ARA may be added to the composition by replacing an equivalent amount of the rest of the overall fat blend normally present in the composition without DHA and ARA.

If utilized, the source of DHA and/or ARA may be any source known in the art such as marine oil, fish oil, single cell oil, egg yolk lipid, and brain lipid. In some embodiments, the DHA and ARA are sourced from single cell Martek oils, DHASCO® and ARASCO®, or variations thereof. The DHA and ARA can be in natural form, provided that the remainder of the LCPUFA source does not result in any substantial deleterious effect on the infant. Alternatively, the DHA and ARA can be used in refined form.

In an embodiment, sources of DHA and ARA are single cell oils as taught in U.S. Pat. Nos. 5,374,567; 5,550,156; and 5,397,591, the disclosures of which are incorporated herein in their entirety by reference. However, the present disclosure is not limited to only such oils.

In some embodiments the nutritional composition may include an enriched lipid fraction derived from milk. In certain embodiments, the enriched lipid fraction comprises an enriched whey protein concentrate (eWPC). The enriched lipid fraction derived from milk may be produced by any number of fractionation techniques. These techniques include but are not limited to melting point fractionation, organic solvent fractionation, super critical fluid fractionation, and any variants and combinations thereof. In some embodiments the nutritional composition may include an enriched lipid fraction derived from milk that contains milk fat globules. Alternatively, eWPC is available commercially, including under the trade name Lacprodan MFGM-10. Another suitable enriched milk product is available commercially under the trade name Lacprodan PL-20. Lacprodan MFGM-10 and Lacprodan PL-20 are available from Arla Food Ingredients of Viby, Denmark. With the addition of an enriched milk product, the lipid composition of infant formulas and other pediatric nutritional compositions can more closely resemble that of human milk. For instance, the theoretical values of phospholipids (mg/L) and gangliosides (mg/L) in an exemplary infant formula which includes Lacprodan MFGM-10 or Lacprodan PL-20 can be calculated as shown in Table 1:

TABLE 1

| | Total milk | | | | | | Other | |
|---|---|---|---|---|---|---|---|---|
| Item | PL | SM | PE | PC | PI | PS | PL | GD3 |
| MFGM-10 | 330 | 79.2 | 83.6 | 83.6 | 22 | 39.6 | 22 | 10.1 |
| PL-20 | 304 | 79 | 64 | 82 | 33 | 33 | 12.2 | 8.5 |

PL: phospholipids; SM: sphingomyelin; PE: phosphatidyl ethanolamine; PC: phosphatidyl choline; PI: phosphatidyl inositol; PS: phosphatidyl serine; GD3: ganglioside GD3.

In some embodiments, the enriched lipid fraction is included in the nutritional composition of the present disclosure at a level of about 0.5 grams per liter (g/L) to about 10 g/L; in other embodiments, the enriched milk product is present at a level of about 1 g/L to about 9 g/L. In still other embodiments, enriched lipid fraction is present in the nutritional composition at a level of about 3 g/L to about 8 g/L. Alternatively, in certain embodiments, the enriched lipid fraction is included in the nutritional composition of the present disclosure at a level of about 0.06 grams per 100 kcal (g/100 kcal) to about 1.5 g/100 kcal; in other embodiments, the enriched lipid fraction is present at a level of about 0.3 g/100 kcal to about 1.4 g/100 kcal. In still other embodiments, the enriched lipid fraction product is present in the nutritional composition at a level of about 0.4 g/100 kcal to about 1 g/100 kcal.

In certain embodiments, the addition of the enriched lipid fraction or the enriched lipid fraction including milk fat globules may provide a source of saturated fatty acids, trans-fatty acids, monounsaturated fatty acids, polyunsaturated fatty acids, BCFAs, CLA, cholesterol, phospholipids, and/or milk fat globule membrane proteins to the nutritional composition.

The milk fat globules may have an average diameter (volume-surface area average diameter) of at least about 2 μm. In some embodiments, the average diameter is in the range of from about 2 μm to about 13 μm. In other embodiments, the milk fat globules may range from about 2.5 μm to about 10 μm. Still in other embodiments, the milk fat globules may range in average diameter from about 3 μm to about 6 μm. The specific surface area of the globules is, in certain embodiments, less than 3.5 m²/g, and in other embodiments is between about 0.9 m²/g to about 3 m²/g. Without being bound by any particular theory, it is believed that milk fat globules of the aforementioned sizes are more accessible to lipases therefore leading to better digestion lipid digestion.

In some embodiments the enriched lipid fraction and/or milk fat globules contain saturated fatty acids. The saturated fatty acids may be present in a concentration from about 0.1 g/100 kcal to about 8.0 g/100 kcal. In certain embodiments the saturated fatty acids may be present from about 0.5 g/100 kcal to about 2.0 g/100 kcal. In still other embodiments the saturated fatty acids may be present from about 3.5 g/100 kcal to about 6.9 g/100 kcal.

Examples of saturated fatty acids suitable for inclusion include, but are not limited to, butyric, valeric, caproic, caprylic, decanoic, lauric, myristic, palmitic, steraic, arachidic, behenic, alignoceric, tetradecanoic, hexadecanoic, palmitic, and octadecanoic acid, and/or combinations and mixtures thereof.

Additionally, the enriched lipid fraction and/or milk fat globules may comprise, in some embodiments, lauric acid. Lauric acid, also known as dodecanoic acid, is a saturated fatty acid with a 12-carbon atom chain and is believed to be one of the main antiviral and antibacterial substances currently found in human breast milk. The milk fat globules may be enriched with triglycerides containing lauric acid at either the Sn-1, Sn-2 and/or Sn-3 positions. Without being bound by any particular theory, it is believed that when the enriched lipid fraction is ingested, the mouth lingual lipase and pancreatic lipase will hydrolyze the triglycerides to a mixture of glycerides including mono-lauric and free lauric acid.

The concentration of lauric acid in the globules varies from 80 mg/100 ml to 800 mg/100 ml The concentration of monolauryl n the globules can be in the range of 20 mg/100 ml to 300 mg/100 ml feed. In some embodiments, the range is 60 mg/100 ml to 130 mg/100 ml.

The enriched lipid fraction and/or milk fat globules may contain trans-fatty acids in certain embodiments. The trans-fatty acids included in the milk fat globules may be monounsaturated or polyunsaturated trans-fatty acids. In some embodiments the trans-fatty acids may be present in an amount from about 0.2 g/100 kcal to about 7.0 g/100 kcal. In other embodiments the trans-fatty acids may be present in an amount from about 3.4 g/100 kcal to about 5.2 g/100 kcal. In yet other embodiments the trans-fatty acids may be present from about 1.2 g/100 kcal to about 4.3 g/100 kcal.

Examples of trans-fatty acids for inclusion include, but are not limited to, vaccenic, or elaidic acid, and mixtures thererof. Moreover, when consumed, mammals convert vaccenic acid into rumenic acid, which is a conjugated linoleic acid that exhibits anticarcinogenic properties. Additionally, a diet enriched with vaccenic acid may help lower total cholesterol, LDL cholesterol and triglyceride levels.

In some embodiments, the enriched lipid fraction and/or milk fat globules may comprise BCFAs. In some embodiments the BCFAs are present at a concentration from about 0.2 g/100 kcal and about 5.82 g/100 kcal. In another embodiment, the BCFAs are present in an amount of from about 2.3 g/100 kcal to about 4.2 g/100 kcal. In yet another embodiment the BCFAs are present from about 4.2 g/100 kcal to about 5.82 g/100 kcal. In still other embodiments, the milk fat globules comprise at least one BCFA.

BCFAs that are identified in human milk are preferred for inclusion in the nutritional composition. Addition of BCFAs to infant or children's formulas allows such formulas to mirror the composition and functionality of human milk and to promote general health and well-being.

In certain embodiments the enriched lipid fraction and/or milk fat globules may comprise CLA. In some embodiments CLA may be present in a concentration from about 0.4 g/100 kcal to about 2.5 g/100 kcal. In other embodiments CLA may be present from about 0.8 g/100 kcal to about 1.2 g/100 kcal. In yet other embodiments CLA may be present from about 1.2 g/100 kcal to about 2.3 g/100 kcal. In still other embodiments, the milk fat globules comprise at least one CLA.

CLAs that are identified in human milk are preferred for inclusion in the nutritional composition. Typically, CLAs are absorbed by the infant from the human milk of a nursing mother. Addition of CLAs to infant or children's formulas allows such formulas to mirror the composition and functionality of human milk and to promote general health and wellbeing.

Examples of CLAs found in the milk fat globules for the nutritional composition include, but are not limited to, cis-9, trans-11 CLA, trans-10, cis-12 CLA, cis-9, trans-12 octadecadienoic acid, and mixtures thereof.

The enriched lipid fraction and/or milk fat globules of the present disclosure comprise monounsaturated fatty acids in some embodiments. The enriched lipid fraction and/or milk fat globules may be formulated to include monounsaturated fatty acids from about 0.8 g/100 kcal to about 2.5 g/100 kcal. In other embodiments the milk fat globules may include monounsaturated fatty acids from about 1.2 g/100 kcal to about 1.8 g/100 kcal.

Examples of monounsaturated fatty acids suitable include, but are not limited to, palmitoleic acid, cis-vaccenic acid, oleic acid, and mixtures thereof.

In certain embodiments, the enriched lipid fraction and/or milk fat globules of the present disclosure comprise polyunsaturated fatty acids from about 2.3 g/100 kcal to about 4.4 g/100 kcal. In other embodiments, the polyunsaturated fatty acids are present from about 2.7 g/100 kcal to about 3.5 g/100 kcal. In yet another embodiment, the polyunsaturated fatty acids are present from about 2.4 g/100 kcal to about 3.3 g/100 kcal.

In some embodiments, the enriched lipid fraction and/or milk fat globules of the present disclosure comprise polyunsaturated fatty acids, such as, for example linoleic acid, linolenic acid, octadecatrienoic acid, arachidonic acid (ARA), eicosatetraenoic acid, eicopsapentaenoic acid (EPA), docosapentaenoic acid (DPA), and docosahexaenoic acid (DHA). Polyunsaturated fatty acids are the precursors for prostaglandins and eicosanoids, which are known to provide numerous health benefits, including, anti-inflammatory response, cholesterol absorption, and increased bronchial function.

The enriched lipid fraction and/or milk fat globules of the present disclosure can also comprise cholesterol in some embodiments from about 100 mg/100 kcal to about 400 mg/100 kal. In another embodiment, cholesterol is present from about 200 mg/100 kcal to about 300 mg/100 kcal. As is similar to human milk and bovine milk, the cholesterol included in the milk fat globules may be present in the outer bilayer membrane of the milk fat globule to provide stability to the globular membrane.

In some embodiments, the enriched lipid fraction and/or milk fat globules of the present disclosure comprise phospholipids from about 50 mg/100 kcal to about 200 mg/100 kcal. In other embodiments, the phospholipids are present from about 75 mg/100 kcal to about 150 mg/100 kcal. In yet other embodiments, the phospholipids are present at a concentration of from about 100 mg/100 kcal to about 250 mg/100 kcal.

In certain embodiments, phospholipids may be incorporated into the milk fat globules to stabilize the milk fat globule by providing a phospholipid membrane or bilayer phospholipid membrane. Therefore, in some embodiments the milk fat globules may be formulated with higher amounts of phospholipids than those found in human milk.

The phospholipid composition of human milk lipids, as the weight percent of total phospholipids, is phosphatidylcholine(“PC”) 24.9%, phosphatidylethanolamine (“PE”) 27.7%, phosphatidylserine (“PS”) 9.3%, phosphatidylinositol (“PI”) 5.4%, and sphingomyelin (“SPGM”) 32.4%, (Harzer, G. et al., Am. J. Clin. Nutr., Vol. 37, pp. 612-621 (1983)). Thus in one embodiment, the milk fat globules comprise one or more of PC, PE, PS, PI, SPGM, and mixtures thereof. Further, the phospholipid composition included in the milk fat globules may be formulated to provide certain health benefits by incorporating desired phospholipids.

In certain embodiments, the enriched lipid fraction and/or milk fat globules of the present disclosure comprise milk fat globule membrane protein. In some embodiments, the milk fat globule membrane protein is present from about 50 mg/100 kcal to about 500 mg/100 kcal.

Galactolipids may be included, in some embodiments, in the enriched lipid fraction and/or milk fat globules of the present disclosure. For purposes of this disclosure “galactolipids” refer to any glycolipid whose sugar group is galactose. More specifically, galactolipids differ from glycosphingolipids in that they do not have nigtrogen in their composition. Galactolipids play an important role in supporting brain development and overall neuronal health. Additionally, the galactolipids, galactocerebroside and sulfatides constitute about 23% and 4% of total myelin lipid content respectively, and thus may be incorporated into the milk fat globules in some embodiments.

In some embodiments, the nutritional composition(s) of the disclosure may comprise at least one protein source other than lactoferrin (if present). The protein source can be any used in the art, e.g., nonfat milk, whey protein, casein, soy protein, hydrolyzed protein, amino acids, and the like. Bovine milk protein sources useful in practicing the present disclosure include, but are not limited to, milk protein powders, milk protein concentrates, milk protein isolates, nonfat milk solids, nonfat milk, nonfat dry milk, whey protein, whey protein isolates, whey protein concentrates, sweet whey, acid whey, casein, acid casein, caseinate (e.g. sodium caseinate, sodium calcium caseinate, calcium caseinate) and any combinations thereof.

In some embodiments, the proteins of the nutritional composition are provided as intact proteins. In other embodiments, the proteins are provided as a combination of both intact proteins and hydrolyzed proteins. In certain embodiments, the proteins may be partially hydrolyzed or extensively hydrolyzed. In still other embodiments, the protein source comprises amino acids. In yet another embodiment, the protein source may be supplemented with glutamine-containing peptides. In another embodiment, the protein component comprises extensively hydrolyzed protein. In still another embodiment, the protein component of the nutritional composition consists essentially of extensively hydrolyzed protein in order to minimize the occurrence of food allergy. In yet another embodiment, the protein source may be supplemented with glutamine-containing peptides.

Accordingly, in some embodiments, the protein component of the nutritional composition comprises either partially or extensively hydrolyzed protein, such as protein from cow's milk. The hydrolyzed proteins may be treated with enzymes to break down some or most of the proteins that cause adverse symptoms with the goal of reducing allergic reactions, intolerance, and sensitization. Moreover, the proteins may be hydrolyzed by any method known in the art.

The terms “protein hydrolysates” or “hydrolyzed protein” are used interchangeably herein and refer to hydrolyzed proteins, wherein the degree of hydrolysis is may be from about 20% to about 80%, or from about 30% to about 80%, or even from about 40% to about 60%.

When a peptide bond in a protein is broken by enzymatic hydrolysis, one amino group is released for each peptide bond broken, causing an increase in amino nitrogen. It should be noted that even non-hydrolyzed protein would contain some exposed amino groups. Hydrolyzed proteins will also have a different molecular weight distribution than the non-hydrolyzed proteins from which they were formed. The functional and nutritional properties of hydrolyzed proteins can be affected by the different size peptides. A molecular weight profile is usually given by listing the percent by weight of particular ranges of molecular weight (in Daltons) fractions (e.g., 2,000 to 5,000 Daltons, greater than 5,000 Daltons).

In a particular embodiment, the nutritional composition is protein-free and contains free amino acids as a protein equivalent source. In this embodiment, the amino acids may comprise, but are not limited to, histidine, isoleucine, leucine, lysine, methionine, cysteine, phenylalanine, tyrosine, threonine, tryptophan, valine, alanine, arginine, asparagine, aspartic acid, glutamic acid, glutamine, glycine, proline, serine, carnitine, taurine and mixtures thereof. In some embodiments, the amino acids may be branched chain amino acids. In other embodiments, small amino acid peptides may be included as the protein component of the nutritional composition. Such small amino acid peptides may be naturally occurring or synthesized. The amount of free amino acids in the nutritional composition may vary from about 1 to about 5 g/100 kcal. In an embodiment, 100% of the free amino acids have a molecular weight of less than 500 Daltons. In this embodiment, the nutritional formulation may be hypoallergenic.

In an embodiment, the protein source comprises from about 40% to about 85% whey protein and from about 15% to about 60% casein.

In some embodiments, the nutritional composition comprises between about 1 g and about 7 g of a protein and/or protein equivalent source per 100 kcal. In other embodiments, the nutritional composition comprises between about 3.5 g and about 4.5 g of protein or protein equivalent per 100 kcal.

Moreover, the nutritional composition of the present disclosure may comprise at least one starch or starch component. A starch is a carbohydrate composed of two distinct polymer fractions: amylose and amylopectin. Amylose is the linear fraction consisting of α-1,4 linked glucose units. Amylopectin has the same structure as amylose, but some of the glucose units are combined in an α-1,6 linkage, giving rise to a branched structure. Starches generally contain 17-24% amylose and from 76-83% amylopectin. Yet special genetic varieties of plants have been developed that produce starch with unusual amylose to amylopectin ratios. Some plants produce starch that is free of amylose. These mutants produce starch granules in the endosperm and pollen that stain red with iodine and that contain nearly 100% amylopectin. Predominant among such amylopectin producing plants are waxy corn, waxy sorghum and waxy rice starch.

The performance of starches under conditions of heat, shear and acid may be modified or improved by chemical modifications. Modifications are usually attained by introduction of substituent chemical groups. For example, viscosity at high temperatures or high shear can be increased or stabilized by cross-linking with di- or polyfunctional reagents, such as phosphorus oxychloride.

In some instances, the nutritional compositions of the present disclosure comprise at least one starch that is gelatinized or pregelatinized. As is known in the art, gelatinization occurs when polymer molecules interact over a portion of their length to form a network that entraps solvent and/or solute molecules. Moreover, gels form when pectin molecules lose some water of hydration owing to competitive hydration of cosolute molecules. Factors that influence the occurrence of gelation include pH, concentration of cosolutes, concentration and type of cations, temperature and pectin concentration. Notably, LM pectin will gel only in the presence of divalent cations, such as calcium ions. And among LM pectins, those with the lowest degree of esterification have the highest gelling temperatures and the greatest need for divalent cations for crossbridging.

Meanwhile, pregelatinization of starch is a process of precooking starch to produce material that hydrates and swells in cold water. The precooked starch is then dried, for example by drum drying or spray drying. Moreover the starch of the present disclosure can be chemically modified to further extend the range of its finished properties. The nutritional compositions of the present disclosure may comprise at least one pregelatinized starch.

Native starch granules are insoluble in water, but, when heated in water, native starch granules begin to swell when sufficient heat energy is present to overcome the bonding forces of the starch molecules. With continued heating, the granule swells to many times its original volume. The friction between these swollen granules is the major factor that contributes to starch paste viscosity.

The nutritional composition of the present disclosure may comprise native or modified starches, such as, for example, waxy corn starch, waxy rice starch, corn starch, rice starch, potato starch, tapioca starch, wheat starch or any mixture thereof. Generally, common corn starch comprises about 25% amylose, while waxy corn starch is almost totally made up of amylopectin. Meanwhile, potato starch generally comprises about 20% amylose, rice starch comprises an amylose:amylopectin ratio of about 20:80, and waxy rice starch comprises only about 2% amylose. Further, tapioca starch generally comprises about 15% to about 18% amylose, and wheat starch has an amylose content of around 25%.

In some embodiments, the nutritional composition comprises gelatinized and/or pre-gelatinized waxy corn starch. In other embodiments, the nutritional composition comprises gelatinized and/or pre-gelatinized tapioca starch. Other gelatinized or pre-gelatinized starches, such as rice starch or potato starch may also be used.

Additionally, the nutritional compositions of the present disclosure comprise at least one source of pectin. The source of pectin may comprise any variety or grade of pectin known in the art. In some embodiments, the pectin has a degree of esterification of less than 50% and is classified as low methylated (“LM”) pectin. In some embodiments, the pectin has a degree of esterification of greater than or equal to 50% and is classified as high-ester or high methylated (“HM”) pectin. In still other embodiments, the pectin is very low (“VL”) pectin, which has a degree of esterification that is less than approximately 15%. Further, the nutritional composition of the present disclosure may comprise LM pectin, HM pectin, VL pectin, or any mixture thereof. The nutritional composition may include pectin that is soluble in water. And, as known in the art, the solubility and viscosity of a pectin solution are related to the molecular weight, degree of esterification, concentration of the pectin preparation and the pH and presence of counterions.

Moreover, pectin has a unique ability to form gels. Generally, under similar conditions, a pectin's degree of gelation, the gelling temperature, and the gel strength are proportional to one another, and each is generally proportional to the molecular weight of the pectin and inversely proportional to the degree of esterification. For example, as the pH of a pectin solution is lowered, ionization of the carboxylate groups is repressed, and, as a result of losing their charge, saccharide molecules do not repel each other over their entire length. Accordingly, the polysaccharide molecules can associate over a portion of their length to form a gel. Yet pectins with increasing degrees of methylation will gel at somewhat higher pH because they have fewer carboxylate anions at any given pH. (J. N. Bemiller, An Introduction to Pectins: Structure and Properties, Chemistry and Function of Pectins; Chapter 1; 1986.)

The nutritional composition may comprise a gelatinized and/or pregelatinized starch together with pectin and/or gelatinized pectin. While not wishing to be bound by this or any other theory, it is believed that the use of pectin, such as LM pectin, which is a hydrocolloid of large molecular weight, together with starch granules, provides a synergistic effect that increases the molecular internal friction within a fluid matrix. The carboxylic groups of the pectin may also interact with calcium ions present in the nutritional composition, thus leading to an increase in viscosity, as the carboxylic groups of the pectin form a weak gel structure with the calcium ion(s), and also with peptides present in the nutritional composition. In some embodiments, the nutritional composition comprises a ratio of starch to pectin that is between about 12:1 and 20:1, respectively. In other embodiments, the ratio of starch to pectin is about 17:1. In some embodiments, the nutritional composition may comprise between about 0.05 and about 2.0% w/w pectin. In a particular embodiment, the nutritional composition may comprise about 0.5% w/w pectin.

Pectins for use herein typically have a peak molecular weight of 8,000 Daltons or greater. The pectins of the present disclosure have a preferred peak molecular weight of between 8,000 and about 500,000, more preferred is between about 10,000 and about 200,000 and most preferred is between about 15,000 and about 100,000 Daltons. In some embodiments, the pectin of the present disclosure may be hydrolyzed pectin. In certain embodiments, the nutritional composition comprises hydrolyzed pectin having a molecular weight less than that of intact or unmodified pectin. The hydrolyzed pectin of the present disclosure can be prepared by any means known in the art to reduce molecular weight. Examples of said means are chemical hydrolysis, enzymatic hydrolysis and mechanical shear. A preferred means of reducing the molecular weight is by alkaline or neutral hydrolysis at elevated temperature. In some embodiments, the nutritional composition comprises partially hydrolyzed pectin. In certain embodiments, the partially hydrolyzed pectin has a molecular weight that is less than that of intact or unmodified pectin but more than 3,300 Daltons.

The nutritional composition may contain at least one acidic polysaccharide. An acidic polysaccharide, such as negatively charged pectin, may induce an anti-adhesive effect on pathogens in a subject's gastrointestinal tract. Indeed, nonhuman milk acidic oligosaccharides derived from pectin are able to interact with the epithelial surface and are known to inhibit the adhesion of pathogens on the epithelial surface.

In some embodiments, the nutritional composition comprises at least one pectin-derived acidic oligosaccharide. Pectin-derived acidic oligosaccharide(s) (pAOS) result from enzymatic pectinolysis, and the size of a pAOS depends on the enzyme use and on the duration of the reaction. In such embodiments, the pAOS may beneficially affect a subject's stool viscosity, stool frequency, stool pH and/or feeding tolerance. The nutritional composition of the present disclosure may comprise between about 2 g pAOS per liter of formula and about 6 g pAOS per liter of formula. In an embodiment, the nutritional composition comprises about 0.2 g pAOS/dL, corresponding to the concentration of acidic oligosaccharides in human milk. (Fanaro et al., “Acidic Oligosaccharides from Pectin Hydrolysate as New Component for Infant Formulae: Effect on Intestinal Flora, Stool Characteristics, and pH”, Journal of Pediatric Gastroenterology and Nutrition, 41: 186-190, August 2005)

In some embodiments, the nutritional composition comprises up to about 20% w/w of a mixture of starch and pectin. In some embodiments, the nutritional composition comprises up to about 19% starch and up to about 1% pectin. In other embodiments, the nutritional composition comprises about up to about 15% starch and up to about 5% pectin. In still other embodiments, the nutritional composition comprises up to about 18% starch and up to about 2% pectin. In some embodiments the nutritional composition comprises between about 0.05% w/w and about 20% w/w of a mixture of starch and pectin. Other embodiments include between about 0.05% and about 19% w/w starch and between about 0.05% and about 1% w/w pectin. Further, the nutritional composition may comprise between about 0.05% and about 15% w/w starch and between about 0.05% and about 5% w/w pectin.

In some embodiments, the nutritional composition comprises at least one additional carbohydrate source, that is, a carbohydrate component provided in addition to the aforementioned starch component. Additional carbohydrate sources can be any used in the art, e.g., lactose, glucose, fructose, corn syrup solids, maltodextrins, sucrose, starch, rice syrup solids, and the like. The amount of the additional carbohydrate component in the nutritional composition typically can vary from between about 5 g and about 22 g/100 kcal. In some embodiments, the amount of carbohydrate is between about 6 g and about 16 g/100 kcal. In other embodiments, the amount of carbohydrate is between about 12 g and about 14 g/100 kcal. In some embodiments, corn syrup solids are preferred. Moreover, hydrolyzed, partially hydrolyzed, and/or extensively hydrolyzed carbohydrates may be desirable for inclusion in the nutritional composition due to their easy digestibility. Specifically, hydrolyzed carbohydrates are less likely to contain allergenic epitopes.

Non-limiting examples of carbohydrate materials suitable for use herein include hydrolyzed or intact, naturally or chemically modified, starches sourced from corn, tapioca, rice or potato, in waxy or non-waxy forms. Non-limiting examples of suitable carbohydrates include various hydrolyzed starches characterized as hydrolyzed cornstarch, maltodextrin, maltose, corn syrup, dextrose, corn syrup solids, glucose, and various other glucose polymers and combinations thereof. Non-limiting examples of other suitable carbohydrates include those often referred to as sucrose, lactose, fructose, high fructose corn syrup, indigestible oligosaccharides such as fructo-oligosaccharides and combinations thereof.

In one particular embodiment, the additional carbohydrate component of the nutritional composition is comprised of 100% lactose. In another embodiment, the additional carbohydrate component comprises between about 0% and 60% lactose. In another embodiment, the additional carbohydrate component comprises between about 15% and 55% lactose. In yet another embodiment, the additional carbohydrate component comprises between about 20% and 30% lactose. In these embodiments, the remaining source of carbohydrates may be any carbohydrate known in the art. In an embodiment, the carbohydrate component comprises about 25% lactose and about 75% corn syrup solids.

In some embodiments the nutritional composition comprises sialic acid. Sialic acids are a family of over 50 members of 9-carbon sugars, all of which are derivatives of neuroaminic acid. The predominant sialic acid family found in humans is from the N-acetylneuraminic acid sub-family. Sialic acids are found in milk, such as bovine and caprine. In mammals, neuronal cell membranes have the highest concentration of sialic acid compared to other body cell membranes. Sialic acid residues are also components of gangliosides.

If included in the nutritional composition, sialic acid may be present in an amount from about 0.5 mg/100 kcal to about 45 mg/100 kcal. In some embodiments sialic acid may be present in an amount from about 5 mg/100 kcal to about 30 mg/100 kcal. In still other embodiments, sialic acid may be present in an amount from about 10 mg/100 kcal to about 25 mg/100 kcal.

As noted, the disclosed nutritional composition may comprise a source of B-glucan. Glucans are polysaccharides, specifically polymers of glucose, which are naturally occurring and may be found in cell walls of bacteria, yeast, fungi, and plants. Beta glucans (β-glucans) are themselves a diverse subset of glucose polymers, which are made up of chains of glucose monomers linked together via beta-type glycosidic bonds to form complex carbohydrates.

β-1,3-glucans are carbohydrate polymers purified from, for example, yeast, mushroom, bacteria, algae, or cereals. (Stone B A, Clarke A E. Chemistry and Biology of (1-3)-Beta-Glucans. London:Portland Press Ltd; 1993.) The chemical structure of β-1,3-glucan depends on the source of the β-1,3-glucan. Moreover, various physiochemical parameters, such as solubility, primary structure, molecular weight, and branching, play a role in biological activities of β-1,3-glucans. (Yadomae T., Structure and biological activities of fungal beta-1,3-glucans. Yakugaku Zasshi. 2000;120:413-431.) β-1,3-glucans are naturally occurring polysaccharides, with or without β-1,6-glucose side chains that are found in the cell walls of a variety of plants, yeasts, fungi and bacteria. β-1,3;1,6-glucans are those containing glucose units with (1,3) links having side chains attached at the (1,6) position(s). P-1,3;1,6 glucans are a heterogeneous group of glucose polymers that share structural commonalities, including a backbone of straight chain glucose units linked by a (3-1,3 bond with (3-1,6-linked glucose branches extending from this backbone. While this is the basic structure for the presently described class of β-glucans, some variations may exist. For example, certain yeast β-glucans have additional regions of P(1,3) branching extending from the β(1,6) branches, which add further complexity to their respective structures.

β-glucans derived from baker's yeast, Saccharomyces cerevisiae, are made up of chains of D-glucose molecules connected at the 1 and 3 positions, having side chains of glucose attached at the 1 and 6 positions. Yeast-derived β-glucan is an insoluble, fiber-like, complex sugar having the general structure of a linear chain of glucose units with a β-1,3 backbone interspersed with β-1,6 side chains that are generally 6-8 glucose units in length. More specifically, β-glucan derived from baker's yeast is poly-(1,6)-β-D-glucopyranosyl-(1,3)-β-D-glucopyranose.

Furthermore, β-glucans are well tolerated and do not produce or cause excess gas, abdominal distension, bloating or diarrhea in pediatric subjects. Addition of (3-glucan to a nutritional composition for a pediatric subject, such as an infant formula, a growing-up milk or another children's nutritional product, will improve the subject's immune response by increasing resistance against invading pathogens and therefore maintaining or improving overall health.

The nutritional composition of the present disclosure comprises β-glucan. In some embodiments, the β-glucan is β-1,3;1,6-glucan. In some embodiments, the (3-1,3;1,6-glucan is derived from baker's yeast. The nutritional composition may comprise whole glucan particle β-glucan, particulate β-glucan, PGG-glucan (poly-1,6-β-D-glucopyranosyl-1,3-β-D-glucopyranose) or any mixture thereof.

In some embodiments, the amount of β-glucan present in the composition is at between about 0.010 and about 0.080 g per 100 g of composition. In other embodiments, the nutritional composition comprises between about 10 and about 30 mg β-glucan per serving. In another embodiment, the nutritional composition comprises between about 5 and about 30 mg β-glucan per 8 fl. oz. (236.6 mL) serving. In other embodiments, the nutritional composition comprises an amount of β-glucan sufficient to provide between about 15 mg and about 90 mg β-glucan per day. The nutritional composition may be delivered in multiple doses to reach a target amount of β-glucan delivered to the subject throughout the day.

In some embodiments, the amount of β-glucan in the nutritional composition is between about 3 mg and about 17 mg per 100 kcal. In another embodiment the amount of β-glucan is between about 6 mg and about 17 mg per 100 kcal.

One or more vitamins and/or minerals may also be added in to the nutritional composition in amounts sufficient to supply the daily nutritional requirements of a subject. It is to be understood by one of ordinary skill in the art that vitamin and mineral requirements will vary, for example, based on the age of the child. For instance, an infant may have different vitamin and mineral requirements than a child between the ages of one and thirteen years. Thus, the embodiments are not intended to limit the nutritional composition to a particular age group but, rather, to provide a range of acceptable vitamin and mineral components.

The nutritional composition may optionally include, but is not limited to, one or more of the following vitamins or derivations thereof: vitamin B₁ (thiamin, thiamin pyrophosphate, TPP, thiamin triphosphate, TTP, thiamin hydrochloride, thiamin mononitrate), vitamin B₂ (riboflavin, flavin mononucleotide, FMN, flavin adenine dinucleotide, FAD, lactoflavin, ovoflavin), vitamin B₃ (niacin, nicotinic acid, nicotinamide, niacinamide, nicotinamide adenine dinucleotide, NAD, nicotinic acid mononucleotide, NicMN, pyridine-3-carboxylic acid), vitamin B₃-precursor tryptophan, vitamin B₆ (pyridoxine, pyridoxal, pyridoxamine, pyridoxine hydrochloride), pantothenic acid (pantothenate, panthenol), folate (folic acid, folacin, pteroylglutamic acid), vitamin B₁₂ (cobalamin, methylcobalamin, deoxyadenosylcobalamin, cyanocobalamin, hydroxycobalamin, adenosylcobalamin), biotin, vitamin C (ascorbic acid), vitamin A (retinol, retinyl acetate, retinyl palmitate, retinyl esters with other long-chain fatty acids, retinal, retinoic acid, retinol esters), vitamin D (calciferol, cholecalciferol, vitamin D3, 1,25,-dihydroxyvitamin D), vitamin E (α-tocopherol, α-tocopherol acetate, α-tocopherol succinate, α-tocopherol nicotinate, α-tocopherol), vitamin K (vitamin K₁, phylloquinone, naphthoquinone, vitamin K₂, menaquinone-7, vitamin K₃, menaquinone-4, menadione, menaquinone-8, menaquinone-8H, menaquinone-9, menaquinone-9H, menaquinone-10, menaquinone-11, menaquinone-12, menaquinone-13), choline, inositol, β-carotene and any combinations thereof.

Further, the nutritional composition may optionally include, but is not limited to, one or more of the following minerals or derivations thereof: boron, calcium, calcium acetate, calcium gluconate, calcium chloride, calcium lactate, calcium phosphate, calcium sulfate, chloride, chromium, chromium chloride, chromium picolonate, copper, copper sulfate, copper gluconate, cupric sulfate, fluoride, iron, carbonyl iron, ferric iron, ferrous fumarate, ferric orthophosphate, iron trituration, polysaccharide iron, iodide, iodine, magnesium, magnesium carbonate, magnesium hydroxide, magnesium oxide, magnesium stearate, magnesium sulfate, manganese, molybdenum, phosphorus, potassium, potassium phosphate, potassium iodide, potassium chloride, potassium acetate, selenium, sulfur, sodium, docusate sodium, sodium chloride, sodium selenate, sodium selenite, sodium molybdate, zinc, zinc oxide, zinc sulfate and mixtures thereof. Non-limiting exemplary derivatives of mineral compounds include salts, alkaline salts, esters and chelates of any mineral compound.

The minerals can be added to nutritional compositions in the form of salts such as calcium phosphate, calcium glycerol phosphate, sodium citrate, potassium chloride, potassium phosphate, magnesium phosphate, ferrous sulfate, zinc sulfate, cupric sulfate, manganese sulfate, and sodium selenite. Additional vitamins and minerals can be added as known within the art.

In an embodiment, the nutritional composition may contain between about 10 and about 50% of the maximum dietary recommendation for any given country, or between about 10 and about 50% of the average dietary recommendation for a group of countries, per serving of vitamins A, C, and E, zinc, iron, iodine, selenium, and choline. In another embodiment, the children's nutritional composition may supply about 10 -30% of the maximum dietary recommendation for any given country, or about 10-30% of the average dietary recommendation for a group of countries, per serving of B-vitamins. In yet another embodiment, the levels of vitamin D, calcium, magnesium, phosphorus, and potassium in the children's nutritional product may correspond with the average levels found in milk. In other embodiments, other nutrients in the children's nutritional composition may be present at about 20% of the maximum dietary recommendation for any given country, or about 20% of the average dietary recommendation for a group of countries, per serving.

The nutritional compositions of the present disclosure may optionally include one or more of the following flavoring agents, including, but not limited to, flavored extracts, volatile oils, cocoa or chocolate flavorings, peanut butter flavoring, cookie crumbs, vanilla or any commercially available flavoring. Examples of useful flavorings include, but are not limited to, pure anise extract, imitation banana extract, imitation cherry extract, chocolate extract, pure lemon extract, pure orange extract, pure peppermint extract, honey, imitation pineapple extract, imitation rum extract, imitation strawberry extract, or vanilla extract; or volatile oils, such as balm oil, bay oil, bergamot oil, cedarwood oil, cherry oil, cinnamon oil, clove oil, or peppermint oil; peanut butter, chocolate flavoring, vanilla cookie crumb, butterscotch, toffee, and mixtures thereof. The amounts of flavoring agent can vary greatly depending upon the flavoring agent used. The type and amount of flavoring agent can be selected as is known in the art.

The nutritional compositions of the present disclosure may optionally include one or more emulsifiers that may be added for stability of the final product. Examples of suitable emulsifiers include, but are not limited to, lecithin (e.g., from egg or soy), alpha lactalbumin and/or mono- and di-glycerides, and mixtures thereof. Other emulsifiers are readily apparent to the skilled artisan and selection of suitable emulsifier(s) will depend, in part, upon the formulation and final product.

The nutritional compositions of the present disclosure may optionally include one or more preservatives that may also be added to extend product shelf life. Suitable preservatives include, but are not limited to, potassium sorbate, sodium sorbate, potassium benzoate, sodium benzoate, calcium disodium EDTA, and mixtures thereof.

The nutritional compositions of the present disclosure may optionally include one or more stabilizers. Suitable stabilizers for use in practicing the nutritional composition of the present disclosure include, but are not limited to, gum arabic, gum ghatti, gum karaya, gum tragacanth, agar, furcellaran, guar gum, gellan gum, locust bean gum, pectin, low methoxyl pectin, gelatin, microcrystalline cellulose, CMC (sodium carboxymethylcellulose), methylcellulose hydroxypropyl methyl cellulose, hydroxypropyl cellulose, DATEM (diacetyl tartaric acid esters of mono- and diglycerides), dextran, carrageenans, and mixtures thereof.

The disclosed nutritional composition(s) may be provided in any form known in the art, such as a powder, a gel, a suspension, a paste, a solid, a liquid, a liquid concentrate, a reconstituteable powdered milk substitute or a ready-to-use product. The nutritional composition may, in certain embodiments, comprise a nutritional supplement, children's nutritional product, infant formula, or any other nutritional composition designed for an infant or a pediatric subject. Nutritional compositions of the present disclosure include, for example, orally-ingestible, health-promoting substances including, for example, foods, beverages, tablets, capsules and powders. Moreover, the nutritional composition of the present disclosure may be standardized to a specific caloric content, it may be provided as a ready-to-use product, or it may be provided in a concentrated form. In some embodiments, the nutritional composition is in powder form with a particle size in the range of 5 μm to 1500 μm, more preferably in the range of 10 μm to 300 μm.

If the nutritional composition is in the form of a ready-to-use product, the osmolality of the nutritional composition may be between about 100 and about 1100 mOsm/kg water, more typically about 200 to about 700 mOsm/kg water.

Suitable fat or lipid sources for the nutritional composition of the present disclosure may be any known or used in the art, including but not limited to, animal sources, e.g., milk fat, butter, butter fat, egg yolk lipid; marine sources, such as fish oils, marine oils, single cell oils; vegetable and plant oils, such as corn oil, canola oil, sunflower oil, soybean oil, palm olein oil, coconut oil, high oleic sunflower oil, evening primrose oil, rapeseed oil, olive oil, flaxseed (linseed) oil, cottonseed oil, high oleic safflower oil, palm stearin, palm kernel oil, wheat germ oil; medium chain triglyceride oils and emulsions and esters of fatty acids; and any combinations thereof.

The nutritional compositions of the disclosure may provide minimal, partial or total nutritional support. The compositions may be nutritional supplements or meal replacements. The compositions may, but need not, be nutritionally complete. In an embodiment, the nutritional composition of the disclosure is nutritionally complete and contains suitable types and amounts of lipid, carbohydrate, protein, vitamins and minerals. The amount of lipid or fat typically can vary from about 1 to about 7 g/100 kcal. The amount of protein typically can vary from about 1 to about 7 g/100 kcal. The amount of carbohydrate typically can vary from about 6 to about 22 g/100 kcal.

The nutritional composition of the present disclosure may further include at least one additional phytonutrient, that is, another phytonutrient component in addition to the pectin and/or starch components described hereinabove. Phytonutrients, or their derivatives, conjugated forms or precursors, that are identified in human milk are preferred for inclusion in the nutritional composition. Typically, dietary sources of carotenoids and polyphenols are absorbed by a nursing mother and retained in milk, making them available to nursing infants. Addition of these phytonutrients to infant or children's formulas allows such formulas to mirror the composition and functionality of human milk and to promote general health and wellbeing.

For example, in some embodiments, the nutritional composition of the present disclosure may comprise, in an 8 fl. oz. (236.6 mL) serving, between about 80 and about 300 mg anthocyanins, between about 100 and about 600 mg proanthocyanidins, between about 50 and about 500 mg flavan-3-ols, or any combination or mixture thereof. In other embodiments, the nutritional composition comprises apple extract, grape seed extract, or a combination or mixture thereof. Further, the at least one phytonutrient of the nutritional composition may be derived from any single or blend of fruit, grape seed and/or apple or tea extract(s).

For the purposes of this disclosure, additional phytonutrients may be added to a nutritional composition in native, purified, encapsulated and/or chemically or enzymatically-modified form so as to deliver the desired sensory and stability properties. In the case of encapsulation, it is desirable that the encapsulated phytonutrients resist dissolution with water but are released upon reaching the small intestine. This could be achieved by the application of enteric coatings, such as cross-linked alginate and others.

Examples of additional phytonutrients suitable for the nutritional composition include, but are not limited to, anthocyanins, proanthocyanidins, flavan-3-ols (i.e., catechins, epicatechins, etc.), flavanones, flavonoids, isoflavonoids, stilbenoids (i.e. resveratrol, etc.), proanthocyanidins, anthocyanins, resveratrol, quercetin, curcumin, and/or any mixture thereof, as well as any possible combination of phytonutrients in a purified or natural form. Certain components, especially plant-based components of the nutritional compositions may provide a source of phytonutrients.

Some amounts of phytonutrients may be inherently present in known ingredients, such as natural oils, that are commonly used to make nutritional compositions for pediatric subjects. These inherent phytonutrient(s) may be but are not necessarily considered part of the phytonutrient component described in the present disclosure. In some embodiments, the phytonutrient concentrations and ratios as described herein are calculated based upon added and inherent phytonutrient sources. In other embodiments, the phytonutrient concentrations and ratios as described herein are calculated based only upon added phytonutrient sources.

In some embodiments, the nutritional composition comprises anthocyanins, such as, for example, glucosides of aurantinidin, cyanidin, delphinidin, europinidin, luteolinidin, pelargonidin, malvidin, peonidin, petunidin, and rosinidin. These and other anthocyanins suitable for use in the nutritional composition are found in a variety of plant sources. Anthocyanins may be derived from a single plant source or a combination of plant sources. Non-limiting examples of plants rich in anthocyanins suitable for use in the inventive composition include: berries (acai, grape, bilberry, blueberry, lingonberry, black currant, chokeberry, blackberry, raspberry, cherry, red currant, cranberry, crowberry, cloudberry, whortleberry, rowanberry), purple corn, purple potato, purple carrot, red sweet potato, red cabbage, eggplant.

In some embodiments, the nutritional composition of the present disclosure comprises proanthocyanidins, which include but are not limited to flavan-3-ols and polymers of flavan-3-ols (e.g., catechins, epicatechins) with degrees of polymerization in the range of 2 to 11. Such compounds may be derived from a single plant source or a combination of plant sources. Non-limiting examples of plant sources rich in proanthocyanidins suitable for use in the inventive nutritional composition include: grape, grape skin, grape seed, green tea, black tea, apple, pine bark, cinnamon, cocoa, bilberry, cranberry, black currant chokeberry.

Non-limiting examples of flavan-3-ols which are suitable for use in the inventive nutritional composition include catechin, epicatechin, gallocatechin, epigallocatechin, epicatechin gallate, epicatechin-3-gallate, epigallocatechin and gallate. Plants rich in the suitable flavan-3-ols include, but are not limited to, teas, red grapes, cocoa, green tea, apricot and apple.

Certain polyphenol compounds, in particular flavan-3-ols, may improve learning and memory in a human subject by increasing brain blood flow, which is associated with an increase and sustained brain energy/nutrient delivery as well as formation of new neurons. Polyphenols may also provide neuroprotective actions and may increase both brain synaptogenesis and antioxidant capability, thereby supporting optimal brain development in younger children.

Preferred sources of flavan-3-ols for the nutritional composition include at least one apple extract, at least one grape seed extract or a mixture thereof. For apple extracts, flavan-3-ols are broken down into monomers occurring in the range 4% to 20% and polymers in the range 80% to 96%. For grape seed extracts fl avan-3-ols are broken down into monomers (about 46%) and polymers (about 54%) of the total favan-3-ols and total polyphenolic content. Preferred degree of polymerization of polymeric flavan-3-ols is in the range of between about 2 and 11. Furthermore, apple and grape seed extracts may contain catechin, epicatechin, epigallocatechin, epicatechin gallate, epigallocatechin gallate, polymeric proanthocyanidins, stilbenoids (i.e. resveratrol), flavonols (i.e. quercetin, myricetin), or any mixture thereof. Plant sources rich in flavan-3-ols include, but are not limited to apple, grape seed, grape, grape skin, tea (green or black), pine bark, cinnamon, cocoa, bilberry, cranberry, black currant, chokeberry.

If the nutritional composition is administered to a pediatric subject, an amount of flavan-3-ols, including monomeric flavan-3-ols, polymeric flavan-3-ols or a combination thereof, ranging from between about 0.01 mg and about 450 mg per day may be administered. In some cases, the amount of flavan-3-ols administered to an infant or child may range from about 0.01 mg to about 170 mg per day, from about 50 to about 450 mg per day, or from about 100 mg to about 300 mg per day.

In an embodiment of the disclosure, flavan-3-ols are present in the nutritional composition in an amount ranging from about 0.4 to about 3.8 mg/g nutritional composition (about 9 to about 90 mg/100 kcal). In another embodiment, flavan-3-ols are present in an amount ranging from about 0.8 to about 2.5 mg/g nutritional composition (about 20 to about 60 mg/100 kcal).

In some embodiments, the nutritional composition of the present disclosure comprises flavanones. Non-limiting examples of suitable flavanones include butin, eriodictyol, hesperetin, hesperidin, homeriodictyol, isosakuranetin, naringenin, naringin, pinocembrin, poncirin, sakuranetin, sakuranin, steurbin. Plant sources rich in flavanones include, but are not limited to orange, tangerine, grapefruit, lemon, lime. The nutritional composition may be formulated to deliver between about 0.01 and about 150 mg flavanones per day.

Moreover, the nutritional composition may also comprise flavonols. Flavonols from plant or algae extracts may be used. Flavonols, such as ishrhametin, kaempferol, myricetin, quercetin, may be included in the nutritional composition in amounts sufficient to deliver between about 0.01 and 150 mg per day to a subject.

The phytonutrient component of the nutritional composition may also comprise phytonutrients that have been identified in human milk, including but not limited to naringenin, hesperetin, anthocyanins, quercetin, kaempferol, epicatechin, epigallocatechin, epicatechin-gallate, epigallocatechin-gallate or any combination thereof. In certain embodiments, the nutritional composition comprises between about 50 and about 2000 nmol/L epicatechin, between about 40 and about 2000 nmol/L epicatechin gallate, between about 100 and about 4000 nmol/L epigallocatechin gallate, between about 50 and about 2000 nmol/L naringenin, between about 5 and about 500 nmol/L kaempferol, between about 40 and about 4000 nmol/L hesperetin, between about 25 and about 2000 nmol/L anthocyanins, between about 25 and about 500 nmol/L quercetin, or a mixture thereof. Furthermore, the nutritional composition may comprise the metabolite(s) of a phytonutrient or of its parent compound, or it may comprise other classes of dietary phytonutrients, such as glucosinolate or sulforaphane.

In certain embodiments, the nutritional composition comprises carotenoids, such as lutein, zeaxanthin, astaxanthin, lycopene, beta-carotene, alpha-carotene, gamma-carotene, and/or beta-cryptoxanthin. Plant sources rich in carotenoids include, but are not limited to kiwi, grapes, citrus, tomatoes, watermelons, papayas and other red fruits, or dark greens, such as kale, spinach, turnip greens, collard greens, romaine lettuce, broccoli, zucchini, garden peas and Brussels sprouts, spinach, carrots.

Humans cannot synthesize carotenoids, but over 34 carotenoids have been identified in human breast milk, including isomers and metabolites of certain carotenoids. In addition to their presence in breast milk, dietary carotenoids, such as alpha and beta-carotene, lycopene, lutein, zeaxanthin, astaxanthin, and cryptoxanthin are present in serum of lactating women and breastfed infants. Carotenoids in general have been reported to improve cell-to-cell communication, promote immune function, support healthy respiratory health, protect skin from UV light damage, and have been linked to reduced risk of certain types of cancer, and all-cause mortality. Furthermore, dietary sources of carotenoids and/or polyphenols are absorbed by human subjects, accumulated and retained in breast milk, making them available to nursing infants. Thus, addition of phytonutrients to infant formulas or children's products would bring the formulas closer in composition and functionality to human milk.

Flavonoids, as a whole, may also be included in the nutritional composition, as flavonoids cannot be synthesized by humans. Moreover, flavonoids from plant or algae extracts may be useful in the monomer, dimer and/or polymer forms. In some embodiments, the nutritional composition comprises levels of the monomeric forms of flavonoids similar to those in human milk during the first three months of lactation. Although flavonoid aglycones (monomers) have been identified in human milk samples, the conjugated forms of flavonoids and/or their metabolites may also be useful in the nutritional composition. The flavonoids could be added in the following forms: free, glucuronides, methyl glucuronides, sulphates, and methyl sulphates.

The nutritional composition may also comprise isoflavonoids and/or isoflavones. Examples include, but are not limited to, genistein (genistin), daidzein (daidzin), glycitein, biochanin A, formononetin, coumestrol, irilone, orobol, pseudobaptigenin, anagyroidisoflavone A and B, calycosin, glycitein, irigenin, 5-O-methylgenistein, pratensein, prunetin, psi-tectorigenin, retusin, tectorigenin, iridin, ononin, puerarin, tectoridin, derrubone, luteone, wighteone, alpinumisoflavone, barbigerone, di-O-methylalpinumisoflavone, and4′-methyl-alpinumisoflavone. Plant sources rich in isoflavonoids, include, but are not limited to, soybeans, psoralea, kudzu, lupine, fava, chick pea, alfalfa, legumes and peanuts. The nutritional composition may be formulated to deliver between about 0.01 and about 150 mg isoflavones and/or isoflavonoids per day.

In an embodiment, the nutritional composition(s) of the present disclosure comprises an effective amount of choline. Choline is a nutrient that is essential for normal function of cells. It is a precursor for membrane phospholipids, and it accelerates the synthesis and release of acetylcholine, a neurotransmitter involved in memory storage. Moreover, though not wishing to be bound by this or any other theory, it is believed that dietary choline and docosahexaenoic acid (DHA) act synergistically to promote the biosynthesis of phosphatidylcholine and thus help promote synaptogenesis in human subjects. Additionally, choline and DHA may exhibit the synergistic effect of promoting dendritic spine formation, which is important in the maintenance of established synaptic connections. In some embodiments, the nutritional composition(s) of the present disclosure includes an effective amount of choline, which is about 20 mg choline per 8 fl. oz. (236.6 mL) serving to about 100 mg per 8 fl. oz. (236.6 mL) serving.

Moreover, in some embodiments, the nutritional composition is nutritionally complete, containing suitable types and amounts of lipids, carbohydrates, proteins, vitamins and minerals to be a subject's sole source of nutrition. Indeed, the nutritional composition may optionally include any number of proteins, peptides, amino acids, fatty acids, probiotics and/or their metabolic by-products, prebiotics, carbohydrates and any other nutrient or other compound that may provide many nutritional and physiological benefits to a subject. Further, the nutritional composition of the present disclosure may comprise flavors, flavor enhancers, sweeteners, pigments, vitamins, minerals, therapeutic ingredients, functional food ingredients, food ingredients, processing ingredients or combinations thereof.

The present disclosure further provides a method for providing nutritional support to a subject. The method includes administering to the subject an effective amount of the nutritional composition of the present disclosure.

The nutritional composition may be expelled directly into a subject's intestinal tract. In some embodiments, the nutritional composition is expelled directly into the gut. In some embodiments, the composition may be formulated to be consumed or administered enterally under the supervision of a physician and may be intended for the specific dietary management of a disease or condition, such as celiac disease and/or food allergy, for which distinctive nutritional requirements, based on recognized scientific principles, are established by medical evaluation.

The nutritional composition of the present disclosure is not limited to compositions comprising nutrients specifically listed herein. Any nutrients may be delivered as part of the composition for the purpose of meeting nutritional needs and/or in order to optimize the nutritional status in a subject.

In some embodiments, the nutritional composition may be delivered to an infant from birth until a time that matches full-term gestation. In some embodiments, the nutritional composition may be delivered to an infant until at least about three months corrected age. In another embodiment, the nutritional composition may be delivered to a subject as long as is necessary to correct nutritional deficiencies. In yet another embodiment, the nutritional composition may be delivered to an infant from birth until at least about six months corrected age. In yet another embodiment, the nutritional composition may be delivered to an infant from birth until at least about one year corrected age.

In still other embodiments, the disclosed nutritional composition may be delivered to a pregnant or lactating woman to provide colic relief to the child/children of the subject woman. The nutritional composition may be delivered to a woman as a liquid nutritional composition, including a reconstituted powder, or as a capsule or other dosage form suitable for an adult.

The nutritional composition of the present disclosure may be standardized to a specific caloric content, it may be provided as a ready-to-use product, or it may be provided in a concentrated form.

The exact composition of a nutritional composition according to the present disclosure can vary from market-to-market, depending on local regulations and dietary intake information of the population of interest. In some embodiments, nutritional compositions according to the disclosure consist of a milk protein source, such as whole or skim milk, plus added sugar and sweeteners to achieve desired sensory properties, and added vitamins and minerals. The fat composition is typically derived from the milk raw materials. Total protein can be targeted to match that of human milk, cow milk or a lower value. Total carbohydrate is usually targeted to provide as little added sugar, such as sucrose or fructose, as possible to achieve an acceptable taste. Typically, Vitamin A, calcium and Vitamin D are added at levels to match the nutrient contribution of regional cow milk. Otherwise, in some embodiments, vitamins and minerals can be added at levels that provide approximately 20% of the dietary reference intake (DRI) or 20% of the Daily Value (DV) per serving. Moreover, nutrient values can vary between markets depending on the identified nutritional needs of the intended population, raw material contributions and regional regulations.

In certain embodiments, the nutritional composition is hypoallergenic. In other embodiments, the nutritional composition is kosher. In still further embodiments, the nutritional composition is a non-genetically modified product. In an embodiment, the nutritional formulation is sucrose-free. The nutritional composition may also be lactose-free. In other embodiments, the nutritional composition does not contain any medium-chain triglyceride oil. In some embodiments, no carrageenan is present in the composition. In other embodiments, the nutritional composition is free of all gums.

In some embodiments, the disclosure is directed to a staged nutritional feeding regimen for a pediatric subject, such as an infant or child, which includes a plurality of different nutritional compositions according to the present disclosure. Each nutritional composition comprises a hydrolyzed protein, at least one pre-gelatinized starch, and at least one pectin. In certain embodiments, the nutritional compositions of the feeding regimen may also include a source of long chain polyunsaturated fatty acid, at least one prebiotic, an iron source, a source of β-glucan, vitamins or minerals, lutein, zeaxanthin, or any other ingredient described hereinabove. The nutritional compositions described herein may be administered once per day or via several administrations throughout the course of a day.

Further provided herein are methods of manufacturing a nutritional composition, such as an infant formula, including at least one or a combination of the following steps: selecting a LA metabolizing probiotic, selecting a source of protein or protein equivalent, selecting a source of fat, selecting a source of enriched milk product, selecting a source of LCPUFAs, and combining the LA metabolizing probiotic, protein or protein equivalent source, LCPUFA source and fat source to produce a nutritional composition. In some embodiments, the method further comprises the step of selecting certain amounts of each ingredient to be incorporated in specific amount based on a 100 kcal serving of the nutritional composition or based on the weight percentage of the nutritional composition.

In certain embodiments, administration of the nutritional compositions disclosed herein modify the microbiota in the gut and reduce the incidence of colic in target subjects. Accordingly, administering the nutritional compositions disclosed herein to infants, including premature infants, or to pregnant or lactating women, can prevent colic and improve an infant's quality of life and reduce the emotional strain and stress for parents.

In some embodiments, the method is directed to manufacturing a powdered nutritional composition. The term “powdered nutritional composition” as used herein, unless otherwise specified, refers to dry-blended powdered nutritional formulations comprising LA metabolizing probiotic, protein or protein equivalent, fat, enriched milk product and LCPUFAs, which are reconstitutable with an aqueous liquid, and which are suitable for oral administration to a human.

Indeed, in some embodiments, the method comprises the steps of dry-blending selected nutritional powders of the nutrients selected to create a base nutritional powder to which additional selected ingredients, such as probiotic, may be added and further blended with the base nutritional powder. The term “dry-blended” as used herein, unless otherwise specified, refers to the mixing of components or ingredients to form a base nutritional powder or, to the addition of a dry, powdered or granulated component or ingredient to a base powder to form a powdered nutritional formulation. In some embodiments, the base nutritional powder is a milk-based nutritional powder. In some embodiments, the base nutritional powder includes at least one fat and one protein or protein equivalent source. The powdered nutritional formulations may have a caloric density tailored to the nutritional needs of the target subject.

The powdered nutritional compositions may be formulated with sufficient kinds and amounts of nutrients so as to provide a sole, primary, or supplemental source of nutrition, or to provide a specialized powdered nutritional formulation for use in individuals afflicted with specific conditions such as colic. For example, in some embodiments, the nutritional compositions disclosed herein may be suitable for administration to pediatric subjects and infants in order provide exemplary health benefits disclosed herein.

The powdered nutritional compositions provided herein may further comprise other optional ingredients that may modify the physical, chemical, hedonic or processing characteristics of the products or serve as nutritional components when used in the targeted population. Many such optional ingredients are known or otherwise suitable for use in other nutritional products and may also be used in the powdered nutritional compositions described herein, provided that such optional ingredients are safe and effective for oral administration and are compatible with the essential and other ingredients in the selected product form. Non-limiting examples of such optional ingredients include preservatives, antioxidants, emulsifying agents, buffers, additional nutrients as described herein, colorants, flavors, thickening agents and stabilizers, and so forth.

The powdered nutritional compositions of he present disclosure may be packaged and sealed in single or multi-use containers, and then stored under ambient conditions for up to about 36 months or longer, more typically from about 12 to about 24 months. For multi-use containers_{;} these packages can be opened and then covered for repeated use by the ultimate user, provided that the covered package is then stored under ambient conditions (e.g., avoid extreme temperatures) and the contents used within about one month or so.

In some embodiments, the method further comprises the step of placing the nutritional compositions in a suitable package. A suitable package may comprise a container, tub, pouch, sachet, bottle, or any other container known and used in the art for containing nutritional composition. In some embodiments, the package containing the nutritional composition is a plastic container. In some embodiments, the package containing the nutritional composition is a metal, glass, coated or laminated cardboard or paper container. Generally, these types of packaging materials are suitable for use with certain sterilization methods utilized during the manufacturing of nutritional compositions formulated for oral administration.

In some embodiments, the nutritional compositions are packaged in a container. The container for use herein may include any container suitable for use with liquid nutritional products that is also capable of withstanding aseptic processing conditions (e.g., sterilization) as described herein and known to those of ordinary skill in the art. A suitable container may be a single-dose container, or may be a multi-dose resealable, or recloseable container that may or may not have a sealing member, such as a thin foil sealing member located below the cap. Non-limiting examples of such containers include bags, plastic bottles or containers, pouches, metal cans, glass bottles, juice box-type containers, foil pouches, plastic bags sold in boxes, or any other container meeting the above-described criteria. In some embodiments, the container is a resealable multi-dose plastic container. In certain embodiments, the resealable multi-dose plastic container further comprises a foil seal and a plastic resealable cap. In some embodiments, the container may include a direct seal screw cap. In other embodiments, the container may be a flexible pouch.

In some embodiments, the nutritional composition is a liquid nutritional composition and is processed via a “retort packaging” or “retort sterilizing” process. The terms “retort packaging” and “retort sterilizing” are used interchangeably herein, and unless otherwise specified, refer to the common practice of filling a container, most typically a metal can or other similar package, with a nutritional liquid and then subjecting the liquid-filled package to he necessary heat sterilization step, to form a sterilized, retort packaged, nutritional liquid product.

In some embodiments, the nutritional compositions disclosed herein are processed via an acceptable aseptic packaging method. The term “aseptic packaging” as used herein, unless otherwise specified, refers to the manufacture of a packaged product without reliance upon the above-described retort packaging step, wherein the nutritional liquid and package are sterilized separately prior to filling, and then are combined under sterilized or aseptic processing conditions to form a sterilized, aseptically packaged, nutritional liquid product.

Examples are provided to illustrate some embodiments of the nutritional composition of the present disclosure but should not be interpreted as any limitation thereon. Other embodiments within the scope of the claims herein will be apparent to one skilled in the art from the consideration of the specification or practice of the nutritional composition or methods disclosed herein. It is intended that the specification, together with the example, be considered to be exemplary only, with the scope and spirit of the disclosure being indicated by the claims which follow the example.

### EXAMPLE 1

This example illustrates an embodiment of a nutritional composition according to the present disclosure.

### Ingredient List:

Lactose (cow's milk), blend of vegetable oils (palm olein oil, coconut oil, soybean oil and high oleic sunflower oil) (plant), non fat milk powder (cow's milk), whey protein concentrate (cow's milk), galacto-oligosaccharide (cow's milk), polydextrose (plant), minerals (calcium carbonate, calcium phosphate, cupric sulfate, ferrous sulfate, magnesium oxide, manganese sulfate, potassium chloride, potassium citrate, sodium citrate, sodium iodide, sodium selenite, tricalcium phosphate and zinc sulfate), emulsifier (soy lecithin) (plant), single cell oils (Mortierella alpina oil, Crypthecodinium cohnii oil) as sources of arachidonic acid (ARA) and docosahexaenoic acid (DHA), lactoferrin (cow's milk), corn syrup solids (plant), Bifidobacterium breve, vitamins (alpha-tocopheryl acetate, biotin, calcium pantothenate, cholecalciferol, choline chloride, cyanocobalamin, folic acid, niacinamide, phytonadione, pyridoxine hydrochloride, riboflavin, sodium ascorbate, thiamine hydrochloride and vitamin A palmitate), inositol, taurine, nucleotides (adenosine monophosphate, cytidine monophosphate, guanosine monophosphate and uridine monophosphate), L-carnitine and antioxidants (ascorbic acid and ascorbyl palmitate).

### Nutritional Composition:

| | | Nutrient level | Nutrient level |
|---|---|---|---|
| Nutrient | Units | (per 100 kcal) | (per 100 g) |
| Energy* | kcal | 100 | 100 |
| Protein | g | 2.1 | 10.6 |
| Lactoferrin | g | 0.09 | 0.45 |
| Fat | g | 5.3 | 27 |
| Linoleic acid (LA) | mg | 810 | 4100 |
| a-linolenic acid (ALA) | mg | 71 | 360 |
| Arachidonic acid (ARA) | mg | 36 | 180 |
| Docosahexaenoic acid | mg | 17.8 | 90 |
| (DHA) | | | |
| Carbohydrates | g | 11.2 | 57 |
| Lactose | g | 10.1 | 51 |
| Galactooligosaccharides | g | 0.31 | 1.56 |
| Polydextrose | g | 0.31 | 1.56 |
| Sialic acid | mg | 20 | 100 |
| Vitamin A | IU | 280 | 1410 |
| Vitamin D | IU | 62 | 310 |
| Vitamin E | IU | 1.9 | 9.6 |
| Vitamin K | μg | 7.2 | 36 |
| Thiamine | μg | 85 | 430 |
| Riboflavin | μg | 170 | 860 |
| Vitamin B6 | μg | 60 | 300 |
| Vitamin B12 | μg | 0.31 | 1.56 |
| Niacin | μg | 660 | 3300 |
| Folic Acid | μg | 18 | 91 |
| Pantothenic acid | μg | 570 | 2900 |
| Biotin | μg | 2.7 | 13.6 |
| Vitamin C | mg | 18 | 91 |
| Calcium | mg | 79 | 400 |
| Phosphorus | mg | 48 | 240 |
| Magnesium | mg | 8 | 40 |
| Iron | mg | 1 | 5 |
| Zinc | mg | 0.8 | 4 |
| Manganese | μg | 18 | 91 |
| Copper | μg | 65 | 330 |
| Iodine | μg | 17 | 86 |
| Selenium | μg | 2.7 | 13.6 |
| Sodium | mg | 28 | 141 |
| Potassium | mg | 110 | 560 |
| Chloride | mg | 65 | 330 |
| Choline | mg | 24 | 121 |
| Inositol | mg | 8.5 | 43 |
| Taurine | mg | 6 | 30 |
| Carnitine | mg | 2 | 10.1 |

*Bacterial strain: about 1 × 10³ to about 1 × 10¹² cfu/100 kcal of LA metabolizing probiotic

### EXAMPLE 2

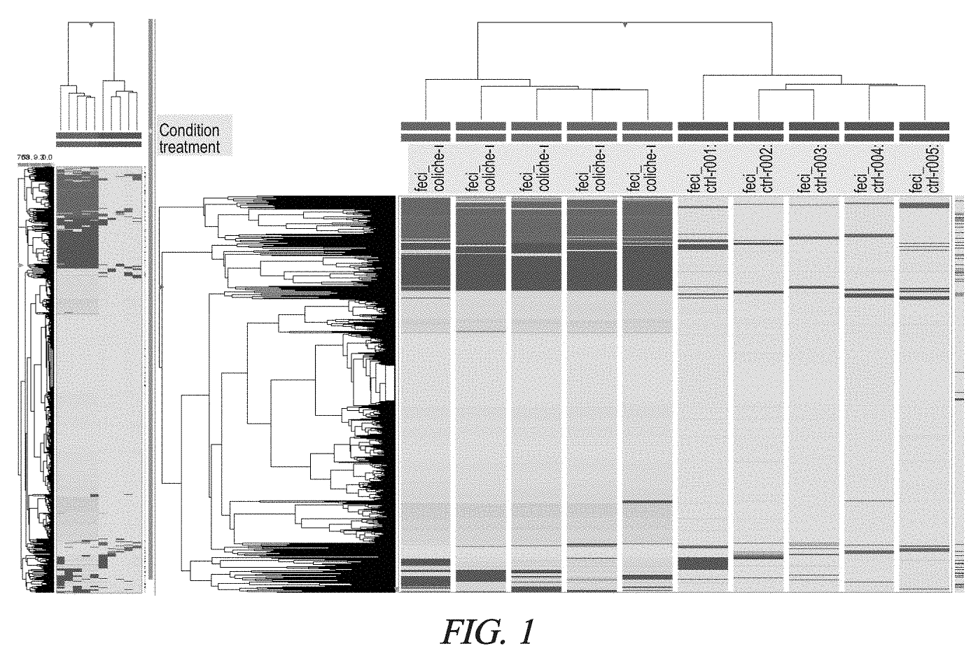

The metabolomics profiles from fecal samples, taken from a single subject during one day with colic and in a day without colic were analyzed and the results provided in FIG. 1. The results show a high presence of undigested lipid compounds in the sample taken during the day with colic as compared to the sample taken in the day without colic.

### EXAMPLE 3

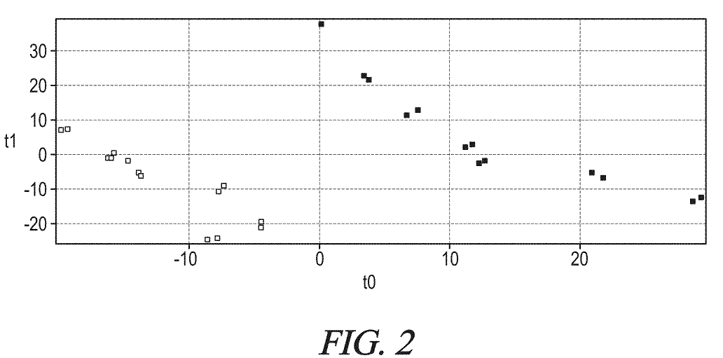

Fourteen (14) fecal samples were obtained from 7 infants without colic (control infants) and from 7 infants with infantile colic (colicky infants). Six out of the seven samples of colicky infants and the 7 control infants were processed by GC-MS chromatography (shown in FIG. 2). The samples were processed in triplicates. The two groups are significantly different for a restrict number of compounds. The compounds are lipids and are involved in the linoleic acid metabolism. The colicky infants and the control infants clustered separately as two different groups for the metabolites present in feces. In particular in the colicky infants LA and Resolvin E are present; both of them index of a possible state of inflammation. The analysis separates colicky infants from control, non-colicky, infants in a distinct way.

### EXAMPLE 4

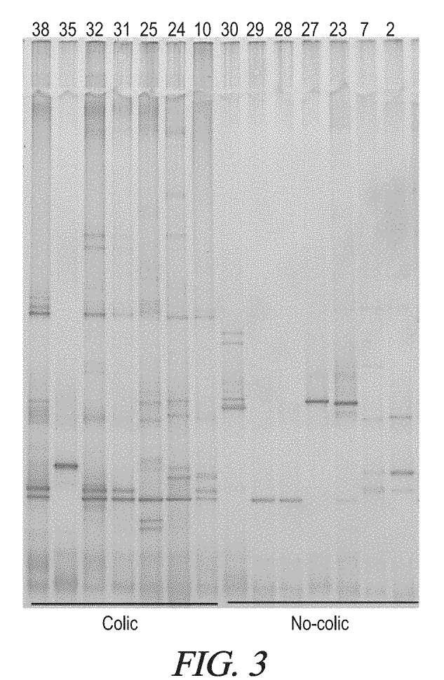

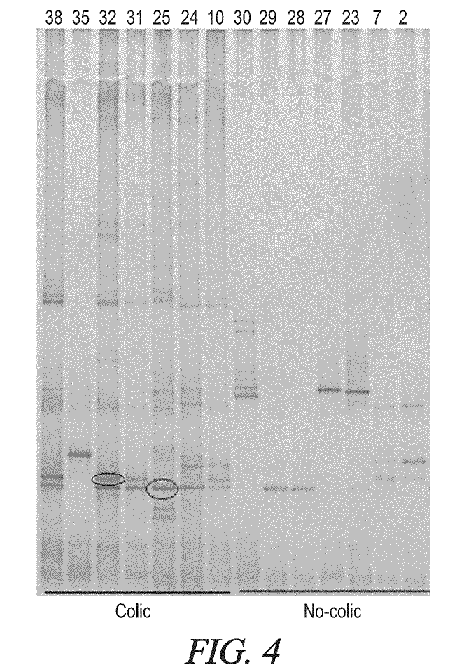

A culture independent approach, namely Denaturing Gradient Gel Electrophoresis (DGGE), was used to study the fecal microbiota composition of the same 14 samples used in Example 3; one of the results is reported in FIG. 3, which reports the DGGE output obtained when a specific bacterial population was analyzed; every band in the gel represents a different population and colicky infants harbor a more complex microbiota compared to control infants. In order to understand the different microorganisms present in the two groups, bands of interest were excised, re-amplified, sequenced (BMR Genomics, Padova, Italy) and then compared by BLAST (Altschul et al., 1997) with sequences in GenBank (http://www.ncbi.nlm.nih.gov/) using the blastn algorithm and in the Ribosomal Database Project (Maidak et al., 1994). The alignment showed that the most prevalent species recovered were Blautia luti, Blautia producta, Blautia wexlerae, Lachnoanaerobaculum orale, Dorea formicigenerans and Ruminococcus gnavus. Blautia is a genus of the Lachnospira. DGGE analysis also indicate that Ruminococcus gnavus and/or Blautia wexlerae were present with prevalence in colicky infants (see FIG. 4).

### EXAMPLE 5

For species-specific quantification of Ruminococcus gnavus, a probe RT-PCR assay with previously described species specific primers and probe F: (5′-TGGCGGCGTGCTTAACA-3′), R:(5′-TCCGAAGAAATCCGTCAAGGT-3′), probe: (FAM-5′-ATGCAAGTCGAGCGAAG-3′-TAMRA) (Joossens et al., 2011) was used on the same 14 samples as Example 3. Template for standard curve was represented by ten-fold dilutions of the genomic DNA of Ruminococcus gnavus ATCC29149. Real-time PCR results indicated that the mean number of 16S rRNA gene copies of the R. gnavus detected in colicky infants was 9.66 ±9.87, and in non-colicky infants 3.55 ±3.36 (log 16S rRNA gene copies of Ruminococcus gnavus per gram of wet feces). These data suggest a difference between the two groups, but the limited number of replicates (7 samples/group) makes it difficult to confirm the outcome with statistical significance.

The quantification the Blautia genus was achieved by using an RT-PCR with primers previously described (Kurakawa et al., 2015). Blautia genus was present at high level in colicky infants compared to control infants, while Ruminococcus gnavus could or could not reach high level in colicky infants. Results then clearly indicated that all colicky infants have higher counts of Blautia spp, some of them have also an high presence of Ruminococcus gnavus while the presence of Bifidobacterium breve is definitely low in colicky infants when compared to non colicky ones. The content of these samples were then analyzed as regards the presence of B. breve, an ecological competitor of the Lachnospiraceae family. Real-time PCR results indicated that the mean number of 16S rRNA gene copies of the Bifidobacterium breve detected in colicky infants was 6.67±6.91, and in non-colicky infants 8.46±8.63 (log 16S rRNA gene copies of Bifidobacterium breve per gram of wet feces); therefore it could be concluded that non colicky infants harbor up to 2 log of Bifidobacterium breve more compared to colicky ones.

### EXAMPLE 6

The 14 fecal samples of Example 3 were processed by means of the Illumina MiSeq protocol. This technique involves the deep sequencing of the V3-V4 region of the 16S rRNA gene of bacteria in the samples, allowing a full coverage of the analyzed diversity, with a correct classification of most sequences up to the species level (Polka et al., 2015). 187,467 sequences were obtained in total, and were downscaled to a common number of 2083 per sample, corresponding to 33,328 sequences in total. Rarefaction curves and coverage analyses showed very good results, with 99.71% of average coverage obtained, indicating that the Illumina analyses captured the largest part of the bacterial diversity in the samples.

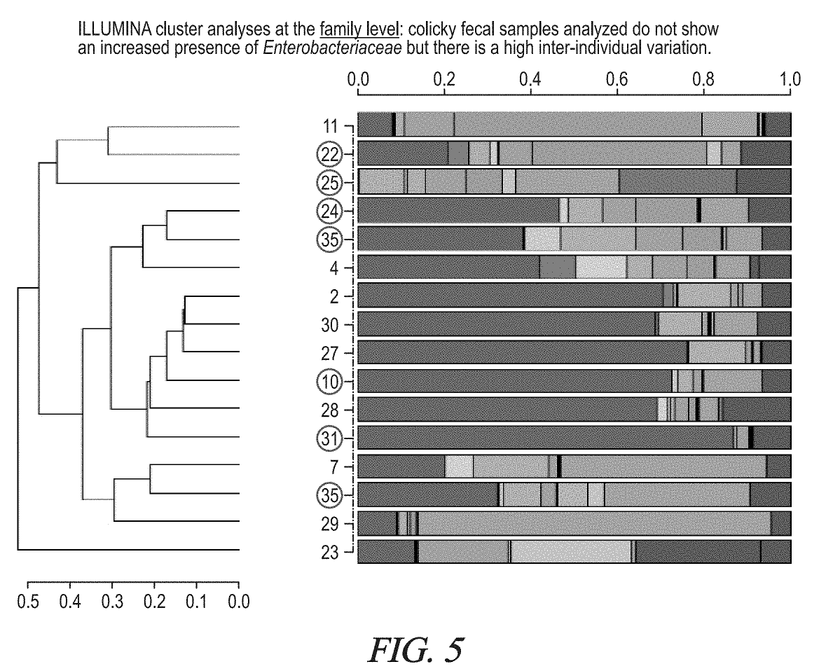

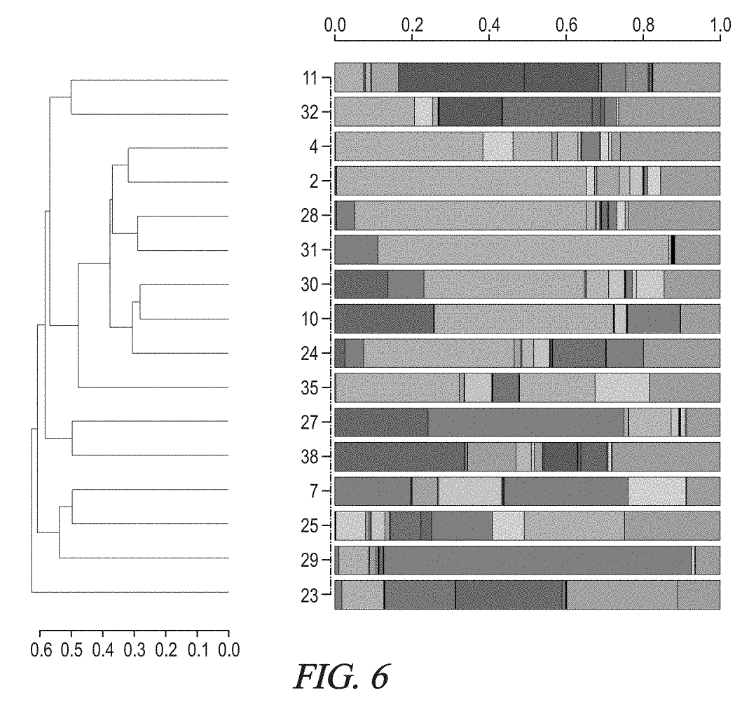

Clustering analyses was carried out, as first step, on the taxonomical results at the family level (FIG. 5). This analysis does not allow a clear cut separation between the colicky and control, non-colicky fecal samples; however it is clear that Enterobacteriaceae (green bars) are not dominant in the colicky babies (colicky samples are marked by a red circle: 10, 24, 25, 31, 32, 35, and 38). The good coverage at the species level of the 14 samples, however, allows to obtain a clearer picture of the microbiota of the two groups (FIG. 6).

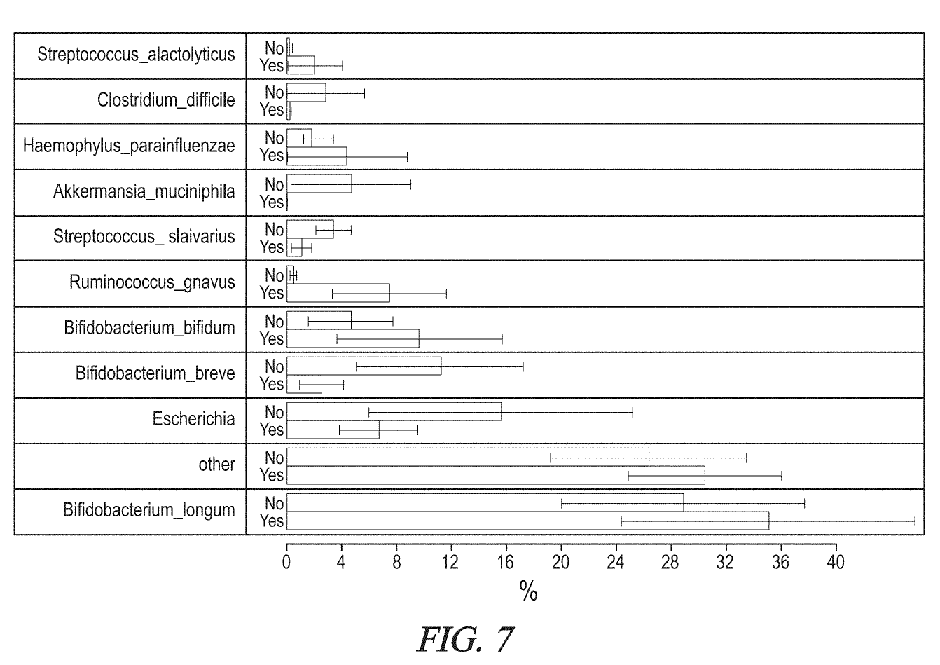

To further confirm at a quantitative level this outcome, a Metastats model was used, which is specifically designed to assess significant differences in the relative abundance of 16S rRNA data originated with high-throughput sequencing methods (Paulson et al., 2011). The model was run on the 10 most abundant species (including Ruminococcus gnavus) representing the 90% of all observed diversity (Metastats is aimed to differentiated species and genera or families of bacteria, this is why in this analysis only Ruminococcus gnavus is taken in consideration): p-value obtained for Ruminococcus gnavus was 0.06, which is near to statistical significance. The graph of differential abundances of these species in the two groups shows indeed a strong increase in the colicky infants (FIG. 7).

The surprising result of all the above studies was that increase of Ruminococcus gnavus in colicky infants is also associated to a decrease of Bifidobacterium breve; both the variations have to occur in order to have colic; these microbial imbalance is also the cause of maldigestion of LA, which is not converted to CLA, adding an additional proinflammatory compounds to the system. This inverse association can explain the appearance of colic as Ruminococcus gnavus and bifidobacteria share the same metabolic pathways for some sugars. The Ruminococcus gnavus, which is able to use intestinal epithelial mucin as a fermentation substrate, and share with B. breve, metabolic pathways for sugar degradation, they are then competitors for the same ecological niche and fermentation substrates.

However, while Bifidobacterium breve does not produce gas and it is able to convert the pro-inflammatory LA into the anti-inflammatory CLA, Ruminococcus gnavus is a hydrogen producing species, unable to convert LA into CLA. Therefore the presence of high levels of Bifidobacterium breve can limit the presence of R. gnavus to levels less harmful for infants.

### EXAMPLE 7

An intervention study has been performed in a colicky, 4 months baby. The baby was breast fed and his diet has been supplemented daily with 10⁹ of a strain of Bifidobacterium breve. Fecal samples were collected before the administration of the probiotic strain during the fussy colic symptoms and after 14 days with the probiotic. Genomic analyses showed that this baby had a high level of Blautia genus (10⁷) while the presence of Ruminococcus gnavus was under the detection limit for the RT-PCR. RT-PCR showed that Bifidobacterium breve was present at the concentration of 10⁸ before the treatment, but it reaches 10⁹ after the treatment. After the treatment with Bifidobacterium breve, the Blautia genus concentration decreased of one logarithm and the presence of Bifidobacterium breve increased by a logarithm.

All references cited in this specification, including without limitation, all papers, publications, patents, patent applications, presentations, texts, reports, manuscripts, brochures, books, internet postings, journal articles, periodicals, and the like, are hereby incorporated by reference into this specification in their entireties. The discussion of the references herein is intended merely to summarize the assertions made by their authors and no admission is made that any reference constitutes prior art. Applicants reserve the right to challenge the accuracy and pertinence of the cited references.

Although embodiments of the disclosure have been described using specific terms, devices, and methods, such description is for illustrative purposes only. The words used are words of description rather than of limitation. It is to be understood that changes and variations may be made by those of ordinary skill in the art without departing from the spirit or the scope of the present disclosure, which is set forth in the following claims. In addition, it should be understood that aspects of the various embodiments may be interchanged in whole or in part. For example, while methods for the production of a commercially sterile liquid nutritional supplement made according to those methods have been exemplified, other uses are contemplated. Therefore, the spirit and scope of the appended claims should not be limited to the description of the versions contained therein.

1. A method for reducing the incidence of colic in a pediatric subject, the method comprising administering to a subject a nutritional composition comprising:
about 1×10³ to about 1×10¹² cfu/100 kcal of LA metabolizing probiotic;
up to about 7 g/100 kcal of a fat or lipid;
up to about 5 g/100 kcal of a protein or protein equivalent source;
about 0.06 g/100 kcal to about 1.5 g/100 kcal of enriched milk product;
about 5 mg/100 kcal to about 90 mg/100 kcal of a source of long chain polyunsaturated fatty acids; and
about 0.015 g/100 kcal to about 1.5 g/100 kcal of a prebiotic composition, wherein the ratio of LA metabolizing probiotic to Ruminococcus gnavus in the gut of the pediatric subject after administration of the nutritional composition is at least 8:1.

2. The method of claim 1, wherein the LA metabolizing probiotic comprises Bifidobacterium breve, Bifidobacterium catenulatum, Bifidobacterium pseudocatenulatum or combinations thereof.

3. The method of claim 1, wherein the source of long chain polyunsaturated fatty acids includes at least one of docosahexaenoic acid, arachidonic acid, and combinations thereof.

4. The method of claim 1, wherein the milk fat globules further comprise gangliosides and phospholipids.

5. The method of claim 1, wherein the nutritional composition comprises at least about 15 mg/100 kcal of lactoferrin from a non-human source.

6. The method of claim 5, wherein lactoferrin is present at a level of about 10 mg/100 kcal to about 200 mg/100 kcal.

7. The method of claim 6, wherein the lactoferrin is bovine lactoferrin.

8. The method of claim 1, wherein polydextrose and galactooligosaccharides comprise at least about 20% of the prebiotic composition.

9. The method of claim 1, wherein the nutritional composition is an infant formula.

10. A nutritional composition for reducing colic in a pediatric subject, comprising:
about 1×10³ to about 1×10¹² cfu/100 kcal of a lactic acid (LA) metabolizing probiotic;
up to about 7 g/100 kcal of a fat or lipid;
up to about 5 g/100 kcal of a protein or protein equivalent source;
about 0.06 g/100 kcal to about 1.5 g/100 kcal of enriched milk product, wherein the enriched milk product comprises from about 0.5% to about 5% sialic acid, from about 2% to about 25% phospholipids, from about 0.4% to about 3% sphingomyelin, from about 0.05% to about 18% gangliosides and from about 0.02% to about 1.2% cholesterol;
about 5 mg/100 kcal to about 90 mg/100 kcal of long chain polyunsaturated fatty acids; and
about 0.015 g/100 kcal to about 1.5 g/100 kcal of a prebiotic composition which comprises polydextrose and a galacto-oligosaccharides.

11. The composition of claim 10, wherein the LA metabolizing probiotic comprises Bifidobacterium breve, Bifidobacterium catenulatum, Bifidobacterium pseudocatenulatum or combinations thereof.

12. The composition of claim 10, wherein the source of long chain polyunsaturated fatty acids is present from about 5 mg/100 kcal to about 75 mg/100 kcal.

13. (canceled)

14. (canceled)

15. (canceled)

16. The composition of claim 10, which further comprises lactoferrin from a non-human source.

17. The composition of claim 16, wherein lactoferrin is present at a level of about 15 mg/100 kcal to about 300 mg/100 kcal.

18. The composition of claim 17, wherein the lactoferrin is bovine lactoferrin.

19. The composition of claim 10, wherein polydextrose and galacto-oligosaccharides comprise at least about 20% of the prebiotic composition.

20. The composition of claim 10, wherein the nutritional composition is an infant formula.

21. The composition of claim 10, wherein the phospholipids comprise at least one phospholipid selected from the group consisting of phosphatidylcholine, phosphatidylethanolamine, phosphatidylserine, phosphatidylinositol, and sphingomyelin.

22. The composition of claim 21, wherein phosphatidylcholine is present in an amount of 24.9% based on the total weight of the phospholipids, wherein phosphatidylethanolamine is present in an amount of about 27.7wt % based on the total weight of the phospholipids, wherein the phosphatidylserine is present in an amount of about 9.3wt % of phosphatidylserine based on the total weight of the phospholipids, wherein the phosphatidylinositol is present in an amount of from about 5.4wt % based on the total weight of the phospholipids, wherein the sphingomyelin is present in an amount of from about 32.4wt % based on the total weight of the phospholipids.
